SEQUENCE LISTING (33 sequences)

SEQ ID NO: 1 - protein, 4 aa, Artificial Sequence
  Description of Artificial Sequencesourcesynthesized
XLGX

SEQ ID NO: 2 - protein, 9 aa, Artificial Sequence
  Description of Artificial Sequencesourcesynthesized
XXXXXXLGX

SEQ ID NO: 3 - protein, 4 aa, Artificial Sequence
  Description of Artificial Sequencesourcesynthesized
SLGI

SEQ ID NO: 4 - protein, 3 aa, Artificial Sequence
  Description of Artificial Sequencesourcesynthesized
XXX

SEQ ID NO: 5 - protein, 15 aa, human
DSENSNFRNEIQSLV

SEQ ID NO: 6 - protein, 15 aa, rat
SISNSRNENEGQSLE

SEQ ID NO: 7 - protein, 15 aa, mouse
STPDTGNENEGQCLE

SEQ ID NO: 8 - protein, 4 aa, Artificial Sequence
  Description of Artificial Sequencesourcesynthesized
ESLV

SEQ ID NO: 9 - protein, 6 aa, Artificial Sequence
  Description of Artificial Sequence sourcesynthesized
TIQSVI

SEQ ID NO: 10 - protein, 8 aa, Artificial Sequence
  Description of Artificial Sequencesourcesynthesized
RGFISSLV

SEQ ID NO: 11 - protein, 8 aa, Artificial Sequence
  Description of Artificial Sequencesourcesynthesized
RETIESTV

SEQ ID NO: 12 - protein, 11 aa, Artificial Sequence
  Description of Artificial Sequencesourcesynthesized
QNFRTYIVSFV

SEQ ID NO: 13 - protein, 13 aa, Artificial Sequence
  Description of Artificial Sequencesourcesynthesized
SDSNMNMNELSEV

SEQ ID NO: 14 - protein, 15 aa, Artificial Sequence
  Description of Artificial Sequencesourcesynthesized
PPTCSQANSGRISTL

SEQ ID NO: 15 - protein, 15 aa, Artificial Sequence
  Description of Artificial Sequencesourcesynthesized
IDLASEFLFLSNSFL

SEQ ID NO: 16 - protein, 15 aa, Artificial Sequence
  Description of Artificial Sequencesourcesynthesized
DSEMYNFRSQLASVV

SEQ ID NO: 17 - protein, 15 aa, Artificial Sequence
  Description of Artificial Sequencesourcesynthesized
IPPDSEDGNEEQSLV

SEQ ID NO: 18 - protein, 4 aa, Artificial Sequence
  Description of Artificial Sequencesourcesynthesized
QSLV

SEQ ID NO: 19 - protein, 5 aa, Artificial Sequence
  Description of Artificial Sequencesource synthesized
IQSLV

SEQ ID NO: 20 - protein, 6 aa, Artificial Sequence
  Description of Artificial Sequencesourcesynthesized
EIQSLV

SEQ ID NO: 21 - protein, 7 aa, Artificial Sequence
  Description of Artificial Sequencesourcesynthesized
NEIQSLV

SEQ ID NO: 22 - protein, 8 aa, Artificial Sequence
  Description of Artificial Sequencesourcesynthesized
RNEIQSLV

SEQ ID NO: 23 - protein, 15 aa, Artificial Sequence
  Description of Artificial Sequencesourcesynthesized
DSENSNFRNEIQSLV

SEQ ID NO: 24 - protein, 427 aa, human
MGAGATGRAMDGPRLLLLLLLGVSLGGAKEACPTGLYTHSGECCKACNLGEGVAQPCGANQTVCEPCLDSVTFSDVVSATEPCKPCTECVGLQSMSAPCVEADDAVCRCAYGYYQDETTGRCEACRVCEAGSGLVFSCQDKQNTVCEECPDGTYSDEANHVDPCLPCTVCEDTERQLRECTRWADAECEEIPGRWITRSTPPEGSDSTAPSTQEPEAPPEQDLIASTVAGVVTTVMGSSQPVVTRGTTDNLIPVYCSILAAVVVGLVAYIAFKRWNSCKQNKGGANSRPVNQTPPPEGEKIHSDSGISVDSQSLHDQQPHTQTASGQALKGDGGLYSSLPPAKREEVEKLLNGSAGDTWRHLAGELGYQPEHIDSFTHEACPVRALLASWATQDSATLDALLAALRRIQRADLVESLCSESTATSPV

SEQ ID NO: 25 - protein, 458 aa, human
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTITLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQENVLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKECQCPHRFQKTCSPI

SEQ ID NO: 26 - protein, 828 aa, human
MNSGVAMKYGNDSSAELSELHSAALASLKGDIVELNKRLQQTEREDLLEKKLAKAQCEQSHLMREHEDVQERTTLRYEERITELHSVIAELNKKIDRLQGTTIREEDEYSELRSELSQSQHEVNEDSRSMDQDQTSVSIPENQSTMVTADMDNCSDINSELQRVLTGLENVVCGRKKSSCSLSVAEVDRHIEQLTTASEHCDLAIKTVEEIEGVLGRDLYPNLAEERSRWEKELAGLREENESLTAMLCSKEEELNRTKATMNAIREERDRLRRRVRELQTRLQSVQATGPSSPGRLTSTNRPINPSTGELSTSSSSNDIPIAKIAERVKLSKTRSESSSSDRPVLGSEISSIGVSSSVAEHLAHSLQDCSNIQEIFQTLYSHGSAISESKIREFEVETERLNSRIEHLKSQNDLLTITLEECKSNAERMSMLVGKYESNATALRLALQYSEQCIEAYELLLALAESEQSLILGQFRAAGVGSSPGDQSGDENITQMLKRAHDCRKTAENAAKALLMKLDGSCGGAFAVAGCSVQPWESLSSNSHTSTTSSTASSCDTEFTKEDEQRLKDYIQQLKNDRAAVKLTMLELESIHIDPLSYDVKPRGDSQRLDLENAVLMQELMAMKEEMAELKAQLYLLEKEKKALELKLSTREAQEQAYLVHIEHLKSEVEEQKEQRMRSLSSTSSGSKDKPGKECADAASPALSLAELRTTCSENELAAEFTNAIRREKKLKARVQELVSALERLTKSSEIRHQQSAEFVNDLKRANSNLVAAYEKAKKKHQNKLKKLESQMMAMVERHETQVRMLKQRIALLEEENSRPHTNETSL

SEQ ID NO: 27 - protein, 672 aa, human
MADVFPGNDSTASQDVANRFARKGALRQKNVHEVKDHKFIARFFKQPTFCSHCTDFIWGFGKGGFQCQVCCFVVHKRCHEFVTFSCPGADKGPDTDDPRSKHKFKIHTYGSPTFCDHCGSLLYGLIHQGMKCDTCDMNVHKQCVINVPSLCGMDHTEKRGRIYLKAEVADEKLHVTVRDAKNLIPMDPNGLSDPYVKLKLIPDPKNESKQKTKTIRSTLNPQWNESFTFKLKPSDKDRRLSVEIWDWDRTTRNDFMGSLSFGVSELMKMPASGWYKLLNQEEGEYYNVPIPEGDEEGNMELRQKFEKAKLGPAGNKVISPSEDRKQPSNNLDRVKLTDFNFLMVLGKGSFGKVMLADRKGTEELYAIKILKKDVVIQDDDVECTMVEKRVLALLDKPPFLTQLHSCFQTVDRLYFVMEYVNGGDLMYHIQQVGKFKEPQAVFYAAEISIGLFFLHKRGIIYRDLKLDNVMLDSEGHIKIADFGMCKEHMMDGVTTRTFCGTPDYIAPEIIAYQPYGKSVDWWAYGVLLYEMLAGQPPFDGEDEDELFQSIMEHNVSYPKSLSKEAVSICKGLMTKHPAKRLGCGPEGERDVREHAFFRRIDWEKLENREIQPPFKPKVCGKGAENFDKFFTRGQPVLTPPDQLVIANIDQSDFEGFSYVNPQFVHPILQSAV

SEQ ID NO: 28 - protein, 471 aa, human
MDILCEENTSLSSTTNSLMQLNDDTRLYSNDFNSGEANTSDAFNWTVDSENRTNLSCEGCLSPSCLSLLHLQEKNWSALLTAVVIILTIAGNILVIMAVSLEKKLQNATNYFLMSLAIADMLLGFLVMPVSMLTILYGYRWPLPSKLCAVWIYLDVLFSTASIMHLCAISLDRYVAIQNPIHHSRFNSRTKAFLKIIAVWTISVGISMPIPVFGLQDDSKVFKEGSCLLADDNFVLIGSFVSFFIPLTIMVITYFLTIKSLQKEATLCVSDLGTRAKLASFSFLPQSSLSSEKLFQRSIHREPGSYTGRRTMQSISNEQKACKVLGIVFFLFVVMWCPFFITNIMAVICKESCNEDVIGALLNVFVWIGYLSSAVNPLVYTLFNKTYRSAFSRYIQCQYKENKKPLQLILVNTIPALAYKSSQLQMGQKKNSKQDAKTTDNDCSMVALGKQHSEEASKDNSDGVNEKVSCV

SEQ ID NO: 29 - protein, 481 aa, human
MALSYRVSELQSTIPEHILQSTFVHVISSNWSGLQTESIPEEMKQIVEEQGNKLHWAALLILMVIIPTIGGNTLVILAVSLEKKLQYATNYFLMSLAVADLLVGLFVMPIALLTIMFEAMWPLPLVLCPAWLFLDVLFSTASIMHLCAISVDRYIAIKKPIQANQYNSRATAFIKITVVWLISIGIAIPVPIKGIETDVDNPNNITCVLTKERFGDFMLFGSLAAFFTPLAIMIVTYFLTIHALQKKAYLVKNKPPQRLTWLTVSTVFQRDETPCSSPEKVAMLDGSRKDKALPNSGDETLMRRTSTIGKKSVQTISNEQRASKVLGIVFFLFLLMWCPFFITNITLVLCDSCNQTTLQMLLEIFVWIGYVSSGVNPLVYTLFNKTFRDAFGRYITCNYRATKSVKTLRKRSSKIYFRNPMAENSKFFKKHGIRNGINPAMYQSPMRLRSSTIQSSSIILLDTLLLTENEGDKTEEQVSYV

SEQ ID NO: 30 - protein, 2843 aa, human
MAAASYDQLLKQVEALKMENSNLRQELEDNSNHLTKLETEASNMKEVLKQLQGSIEDEAMASSGQIDLLERLKELNLDSSNFPGVKLRSKMSLRSYGSREGSVSSRSGECSPVPMGSFPRRGFVNGSRESTGYLEELEKERSLLLADLDKEEKEKDWYYAQLQNLTKRIDSLPLTENFSLQTDMTRRQLEYEARQIRVAMEEQLGTCQDMEKRAQRRIARIQQIEKDILRIRQLLQSQATEAERSSQNKHETGSHDAERQNEGQGVGEINMATSGNGQGSTTRMDHETASVLSSSSTHSAPRRLTSHLGTKVEMVYSLLSMLGTHDKDDMSRTLLAMSSSQDSCISMRQSGCLPLLIQLLHGNDKDSVLLGNSRGSKEARARASAALHNIIHSQPDDKRGRREIRVLHLLEQIRAYCSTCWEWQEAHEPGMDQDKNPMPAPVEHQICPAVCVLMKLSFDEEHRHAMNELGGLQAIAELLQVDCEMYGLTNDHYSITLRRYAGMALTNLTFGDVANKATLCSMKGCMRALVAQLKSESEDLQQVIASVLRNLSWRADVNSKKTLREVGSVKALMECALEVKKESTLKSVLSALWNLSAHCTENKADICAVDGALAFLVGTLTYRSQTNTLAIIESGGGILRNVSSLIATNEDHRQILRENNCLQTLLQHLKSHSLTIVSNACGTLWNLSARNPKDQEALWDMGAVSMLKNLIHSKHKMIAMGSAAALRNLMANRPAKYKDANIMSPGSSLPSLHVRKQKALEAELDAQHLSETFDNIDNISPKASHRSKQRHKQSLYGDYVFDTNRHDDNRSDNFNTGNMTVLSPYLNTTVLPSSSSSRGSLDSSRSEKDRSLERERGIGLGNYHPATENPGTSSKRGLQISTTAAQIAKVMEEVSAIHTSQEDRSSGSTTELHCVTDERNALRRSSAAHTHSNTYNFTKSENSNRTCSMPYAKLEYKRSSNDSLNSVSSSDGYGKRGQMKPSIESYSEDDESKFCSYGQYPADLAHKIHSANHMDDNDGELDTPINYSLKYSDEQLNSGRQSPSQNERWARPKHIIEDEIKQSEQRQSRNQSTTYPVYTESTDDKHLKFQPHFGQQECVSPYRSRGANGSETNRVGSNHGINQNVSQSLCQEDDYEDDKPTNYSERYSEEEQHEEEERPTNYSIKYNEEKRHVDQPIDYSILKATDIPSSQKQSFSFSKSSSGQSSKTEHMSSSSENTSTPSSNAKRQNQLHPSSAQSRSGQPQKAATCKVSSINQETIQTYCVEDTPICFSRCSSLSSLSSAEDEIGCNQTTQEADSANTLQIAEIKEKIGTRSAEDPVSEVPAVSQHPRTKSSRLQGSSLSSESARHKAVEFSSGAKSPSKSGAQTPKSPPEHYVQETPLMFSRCTSVSSLDSFESRSIASSVQSEPCSGMVSGIISPSDLPDSPGQTMPPSRSKTPPPPPQTAQTKREVPKNKAPTAEKRESGPKQAAVNAAVQRVQVLPDADTLLHFATESTPDGFSCSSSLSALSLDEPFIQKDVELRIMPPVQENDNGNETESEQPKESNENQEKEAEKTIDSEKDLLDDSDDDDIEILEECIISAMPTKSSRKAKKPAQTASKLPPPVARKPSQLPVYKLLPSQNRLQPQKHVSFTPGDDMPRVYCVEGTPINFSTATSLSDLTIESPPNELAAGEGVRGGAQSGEFEKRDTIPTEGRSTDEAQGGKTSSVTIPELDDNKAEEGDILAECINSAMPKGKSHKPFRVKKIMDQVQQASASSSAPNKNQLDGKKKKPTSPVKPIPQNTEYRTRVRKNADSKNNLNAERVFSDNKDSKKQNLKNNSKDFNDKLPNNEDRVRGSFAFDSPHHYTPIEGTPYCFSRNDSLSSLDFDDDDVDLSREKAELRKAKENKESEAKVTSHTELTSNQQSANKTQAIAKQPINRGQPKPILQKQSTFPQSSKDIPDRGAATDEKLQNFAIENTPVCFSHNSSLSSLSDIDQENNNKENEPIKETEPPDSQGEPSKPQASGYAPKSFHVEDTPVCFSRNSSLSSLSIDSEDDLLQECISSAMPKKKKPSRLKGDNEKHSPRNMGGILGEDLTLDLKDIQRPDSEHGLSPDSENFDWKAIQEGANSIVSSLHQAAAAACLSRQASSDSDSILSLKSGISLGSPFHLTPDQEEKPFTSNKGPRILKPGEKSTLETKKIESESKGIKGGKKVYKSLITGKVRSNSEISGQMKQPLQANMPSISRGRTMIHIPGVRNSSSSTSPVSKKGPPLKTPASKSPSEGQTATTSPRGAKPSVKSELSPVARQTSQIGGSSKAPSRSGSRDSTPSRPAQQPLSRPIQSPGRNSISPGRNGISPPNKISQLPRTSSPSTASTKSSGSGKMSYTSPGRQMSQQNLTKQTGLSKNASSIPRSESASKGLNQMNNGNGANKKVELSRMSSTKSSGSESDRSERPVLVRQSTFIKEAPSPTLRRKLEESASFESLSPSSRPASPTRSQAQTPVLSPSLPDMSLSTHSSVQAGGWRKLPPNLSPTIEYNDGRPAKRHDIARSHSESPSRLPINRSGTWKREHSKHSSSLPRVSTWRRTGSSSSILSASSESSEKAKSEDEKHVNSISGTKQSKENQVSAKGTWRKIKENEFSPTNSTSQTVSSGATNGAESKTLIYQMAPAVSKTEDVWVRIEDCPINNPRSGRSPTGNTPPVIDSVSEKANPNIKDSKDNQAKQNVGNGSVPMRTVGLENRLNSFIQVDAPDQKGTEIKPGQNNPVPVSETNESSIVERTPFSSSSSSKHSSPSGTVAARVTPFNYNPSPRKSSADSTSARPSQIPTPVNNNTKKRDSKTDSTESSGTQSPKRHSGSYLVTSV

SEQ ID NO: 31 - DNA, 65 bp, Artificial Sequence
  Description of Artificial Sequencesourcesynthesized
cggaattcnnnnnnnnnaacagcnnnnnnnnnaatgaannncaaagtctgnnntgaggatcctca

SEQ ID NO: 32 - DNA, 65 bp, Artificial Sequence
  Description of Artificial Sequencesourcesynthesized
cggaattcgactcagaannnnnnaacttcagannnnnnatcnnnnnnnnngtctgaggatcctca

SEQ ID NO: 33 - DNA, 65 bp, Artificial Sequence
  Description of Artificial Sequencesourcesynthesized
cggaattcnnnnnnnnnnnnnnnnnnnnnnnnnnnnnnnnnnnnnnnnnnnnntgaggatcctca

(19) United States
(12) Patent Application Publication
(10) Pub. No.: US 2003/0203414 A1
(43) Pub. Date: Oct. 30, 2003

(54) COMPOUNDS THAT INHIBIT THE INTERACTION BETWEEN SIGNAL-TRANSDUCING PROTEINS AND THE GLGF (PDZ/DHR) DOMAIN AND USES THEREOF

(76) Inventors: TAKA-AKI SATO, FORT LEE, NJ (US); JUNN YANAGISAWA, TOKYO (JP)

Correspondence Address: JOHN P WHITE, COOPER & DUNHAM, 1185 AVENUE OF THE AMERICAS, NEW YORK, NY, 10036

(21) Appl. No.: 09/230,111

(22) PCT Filed: Jul. 18, 1997
(86) PCT No.: PCT/US97/12677

(51) Int. Cl. G01N 33/53; G01N 33/574; A61K 39/395; A61K 38/08; A61K 38/10; A61K 38/06

(52) U.S. Cl. 435/7.23; 424/155.1; 514/2; 514/14; 514/15; 514/16; 514/17; 514/18

(57) ABSTRACT

This invention provides for a composition capable of inhibiting specific binding between a signal-transducing protein and a cytoplasmic protein. This invention also provides a method of identifying a compound capable of inhibiting specific binding between a signal-transducing protein and a cytoplasmic protein. This invention also provides a method of inhibiting the proliferation of cancer cells. This invention also provides a method of treating cancer with a composition in an amount effective to result in an amount in apoptosis of the cells. This invention also provides a method of inhibiting the proliferation of virally infected cells. This invention also provides for a method of treating a virally-infected subject with a composition in an amount effective to result in apoptosis of the cells. This invention also provides for pharmaceutical compositions.

The invention disclosed herein was made with Government support under Grant No. R01GM55147-01 from the National Institutes of Health of the United States Department of Health and Human Services. Accordingly, the U.S. Government has certain rights in this invention.

### BACKGROUND

Throughout this application, various publications are referenced by author and date. Full citations for these publications may be found listed alphabetically at the end of the specification immediately preceding Sequence Listing and the claims. The disclosures of these publications in their entireties are hereby incorporated by reference into this application in order to more fully describe the state of the art as known to those skilled therein as of the date of the invention described and claimed herein.

Fas (APO-1/CD95) and its ligand have been identified as important signal-mediators of apoptosis (Itoh, et al. 1991) The structural organization of Fas (APO-1/CD95) has suggested that it is a member of the tumor necrosis factor receptor superfamily, which also includes the p75 nerve growth factor receptor (NGFR) (Johnson, et al. 1986), the T-cell-activation marker CD27 (Camerini, et al. 1991), the Hodgkin-lymphoma-associated antigen CD30 (Smith, et al. (1993), the human B cell antigen CD40 (Stamenkovic, et al. 1989), and T cell antigen OX40 (Mallett, et al. 1990). Genetic mutations of both Fas and its ligand have been associated with lymphoproliferative and autoimmune disorders in mice (Watanabe-Fukunaga, et al. 1992; Takahashi, et al. 1994).

Furthermore, alterations of Fas expression level have been thought to lead to the induction of apoptosis in T-cells infected with human immunodeficiency virus (HIV) (Westendorp, et al. 1995).

Several Fas-interacting signal transducing molecules, such as Fas-associated phosphatase-1 (FAP-1) (FIG. 1) (Sato, et al. 1995) FADD/MORT1/CAP-1/CAP-2 (Chinnaiyan, et al. 1995; Boldin, et al. 1995; Kischkel, et al. 1995) and RIP (Stanger, et al. 1995), have been identified using yeast two-hybrid and biochemical approaches. All but FAP-1 associate with the functional cell death domain of Fas and overexpression of FADD/MORT1 or RIP induces apoptosis in cells transfected with these proteins. In contrast, FAP-1 is the only protein that associates with the negative regulatory domain (C-terminal 15 amino acids) (Ito, et al. 1993) of Fas and that inhibits Fas-induced apoptosis.

FAP-1 (PTPN13) has several alternatively-spliced forms that are identical to PTP-BAS/hPTP1E/PTPL1, (Maekawa, et al. 1994; Banville, et al. 1994; Saras, et al. 1994) and contains a membrane-binding region similar to those found in the cytoskeleton-associated proteins, ezrin, (Gould et al. 1989) radixin (Funayama et al. 1991) moesin (Lankes, et al. 1991), neurofibromatosis type II gene product (NFII) (Rouleau, et al. 1993), and protein 4.1 (Conboy, et al. 1991), as well as in the PTPases PTPH1 (Yang, et al. 1991), PTP-MEG (Gu, et al. 1991), and PTPD1 (Vogel, et al. 1993). FAP-1 intriguingly contains six GLGF (PDZ/DHR) repeats that are thought to mediate intra-and inter-molecular interactions among protein domains. The third GLGF repeat of FAP-1 was first identified as a domain showing the specific interaction with the C-terminus of Fas receptor (Sato, et al. 1995). This suggests that the GLGF domain may play an important role in targeting proteins to the submembranous cytoskeleton and/or in regulating biochemical activity. GLGF repeats have been previously found in guanylate kinases, as well as in the rat post-synaptic density protein (PSD-95) (Cho, et al. 1992), which is a homolog of the Drosophila tumor suppressor protein, lethal-(1)-disc-large-1 [dlg-1] (Woods, et al 1991; Kitamura, et al. 1994). These repeats may mediate homo- and hetero-dimerization, which could potentially influence PTPase activity, binding to Fas, and/or interactions of FAP-1 with other signal transduction proteins. Recently, it has also been reported that the different PDZ domains of proteins interact with the C-terminus of ion channels and other proteins (FIG. 1) (TABLE 1)(Kornau, et al. 1995; Kim, et al. 1995; Matsumine, et al. 1996).

TABLE 1
Proteins that interact with PDZ domains.

| | C-terminal | Associated | |
|---|---|---|---|
| Protein | sequence | protein | Reference |
| Fas (APO-1/CD95) | SLV | FAP-1 | 2 |
| NMDA receptor | SDV | PSD95 | 3 |
| NR2 subunit | | | |
| Shaker-type K+ | TDV | PSD95 & DLG | 4 |
| channel | | | |
| APC | TEV | DLG | 5 |

### SUMMARY OF THE INVENTION

This invention provides a composition capable of inhibiting specific binding between a signal-transducing protein and a cytoplasmic protein containing the amino acid sequence (G/S/A/E)-L-G-(F/I/L) (Sequence I.D. No. 1). Further, the cytoplasmic protein may contain the amino acid sequence (K/R/Q)-Xₙ-(G/S/A/E)-L-G-(F/I/L) (Sequence I.D. No. 2), wherein X represents any amino acid which is selected from the group comprising the twenty naturally occurring amino acids and n represents at least 2, but not more than 4. In a preferred embodiment, the amino acid sequence is SLGI (Sequence I.D. No. 3). Further, the invention provides for a composition when the signal-transducing protein has at its carboxyl terminus the amino acid sequence (S/T)-X-(V/I/L) (Sequence I.D. No. 4), wherein each—represents a peptide bond, each parenthesis encloses amino acids which are alternatives to one other, each slash within such parentheses separating the alternative amino acids, and the X represents any amino acid which is selected from the group comprising the twenty naturally occurring amino acids.

This invention also provides for a method of identifying a compound capable of inhibiting specific binding between a signal-transducing protein and a cytoplasmic protein containing the amino acid sequence (G/S/A/E)-L-G-(F/I/L). Further this invention provides for a method of identifying a compound capable of inhibiting specific binding between a signal-transducing protein having at its carboxyl terminus the amino acid sequence (S/T)-X-(V/L/I) and a cytoplasmic protein.

This invention also provides for a method inhibiting the proliferation of cancer cells, specifically, where the cancer cells are derived from organs comprising the colon, liver, breast, ovary, testis, lung, stomach, spleen, kidney, prostate, uterus, skin, head, thymus and neck, or the cells are derived from either T-cells or B-cells.

This invention also provides for a method of treating cancer in a subject in an amount of the composition of effective to result in apoptosis of the cells, specifically, where the cancer cells are derived from organs comprising the thymus, colon, liver, breast, ovary, testis, lung, stomach, spleen, kidney, prostate, uterus, skin, head and neck, or the cells are derived from either T-cells or B-cells.

This invention also provides for a method of inhibiting the proliferation of virally infected cells, specifically wherein the virally infected cells are infected with the Hepatitis B virus, Epstein-Barr virus, influenza virus, Papilloma virus, Adenovirus, Human T-cell lymphtropic virus, type 1 or HIV.

This invention also provides a pharmaceutical composition comprising compositions capable of inhibiting specific binding between a signal-transducing protein and a cytoplasmic protein.

This invention also provides a pharmaceutical composition comprising compounds identified to be capable of inhibiting specific binding between a signal-transducing protein and a cytoplasmic protein.

### BRIEF DESCRIPTION OF THE FIGURES

FIG. 1. Diagram of Fas-associated phosphatase-1 protein, showing the six GLGF (PDZ/DHR) domain repeats; comparison of similar membrane binding sites with other proteins and proteins that contain GLGF (PDZ/DHR) repeats.

FIGS. 2A, 2B, 2C and 2D. Mapping of the minimal region of the C-terminal of Fas required for the binding to FAP-1. Numbers at right show each independent clone (FIGS. 2C and 2D).

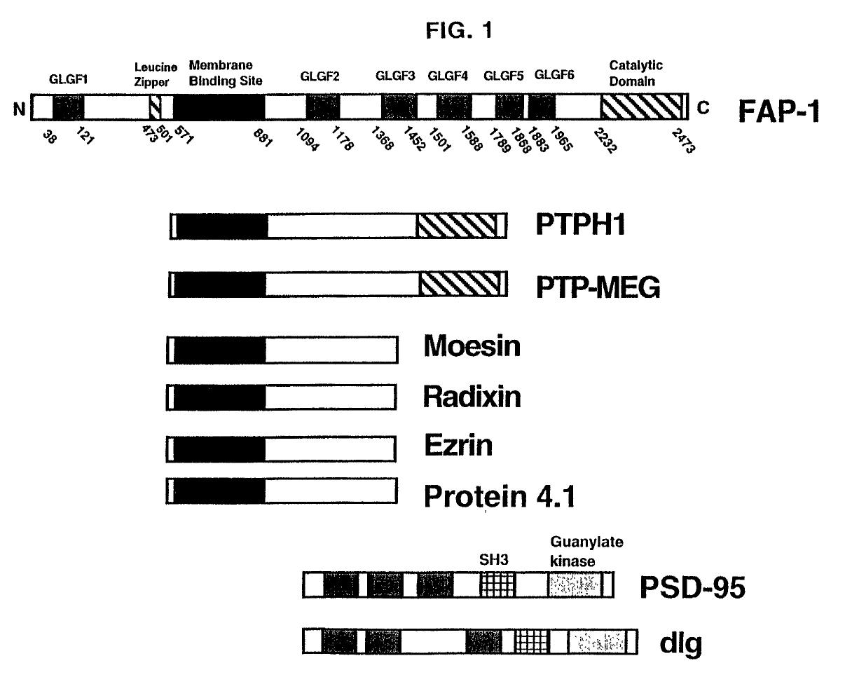

2A. Strategy for screening of a random peptide library by the yeast two-hybrid system.

2E. Alignment of the C-terminal 15 amino acids of Fas between human (Sequence I.D. No. 5), rat (Sequence I.D. No. 6), and mouse (Sequence I.D. No. 7).

2C. The results of screening a semi-random peptide library. Top row indicates the amino acids which were fixed based on the homology between human and rat. Dash lines show unchanged amino acids.

2D. The results of screening a random peptide library (Sequence I.D. No. 8, Sequence I.D. No. 9, Sequence I.D. No. 10, Sequence I.D. No. 11, Sequence I.D. No. 12, Sequence I.D. No. 13, Sequence I.D. No. 14, Sequence I.D. No. 15, Sequence I.D. No. 16, Sequence I.D. No. 17, respectively).

FIGS. 3A, 3B and 3C. Inhibition assay of Fas/FAP-1 binding in vitro.

3A. Inhibition assay of Fas/FAP-1 binding using the C-terminal 15 amino acids of Fas. GST-Fas fusion protein (191-355) was used for in vitro binding assay (lane 1, 3-10). GST-Fas fusion protein (191-320) (lane 2) and 1 mM human PAMP (N-terminal 20 amino acids of proadrenomedullin, M.W. 2460.9) (lane 3) were used as negative controls. The concentrations of the C-terminal 15 amino acids added were 1 (lane 4), 3 (lane 5), 10 (lane 6), 30 (lane 7), 100 (lane 8), 300 (lane 9), and 1000 μM (lane 10).

3B. Inhibition assay of Fas/FAP-1 binding using the truncated peptides corresponding to the C-terminal 15 amino acids of Fas. All synthetic peptides were acetylated for this inhibition assay (Sequence I.D. No. 4, Sequence I.D. No. 18, Sequence I.D. No.: 19, Sequence I.D. No. 20, Sequence I.D. No. 21, Sequence I.D. No. 22, Sequence I.D. No. 23, respectively)

3C. Inhibitory effect of Fas/FAP-1 binding using the scanned tripeptides.

FIGS. 4A, 4B, 4C and 4D.

4A. Interaction of the C-terminal 3 amino acids of Fas with FAP-1 in yeast.

4B. Interaction of the C-terminal 3 amino acids of Fas with FAP-1 in vitro.

4C. Immuno-precipitation of native Fas with GST-FAP-1.

4D. Inhibition of Fas/FAP-1 binding with Ac-SLV or Ac-SLY.

FIGS. 5A, 5B, 5C, 5D, 5E and 5F. Microinjection of Ac-SLV into the DLD-1 cell line. Triangles identify the cells both that were could be microinjected with Ac-SLV and that showed condensed chromatin identified. On the other hand, only one cell of the area appeared apoptotic when microinjected with Ac-SLY.

5A. Representative examples of the cells microinjected with Ac-SLV in the presence of 500 ng/ml CH11 are shown in phase contrast.

5B. Representative examples of the cells microinjected with AC-SLY in the presence of 500 ng/ml CH11 are shown in phase contrast.

5C. Representative examples of the cells microinjected with Ac-SLV in the presence of 500 ng/ml CH11 are shown stained with FITC.

5D. Representative examples of the cells microinjected with AC-SLY in the presence of 500 ng/ml CH11 are shown stained with FITC.

5E. Representative examples of the cells microinjected with Ac-SLV in the presence of 500 ng/ml CH11 are shown with fluorescent DNA staining with Hoechst 33342.

5F. Representative examples of the cells microinjected with AC-SLY in the presence of 500 ng/ml CH11 are shown in fluorescent DNA staining with Hoechst 33342.

FIG. 6. Quantitation of apoptosis in microinjected DLD-1 cells.

FIGS. 7A, 7B, 7C, 7D, 7E, 7F, 7G, and 7H.

7A. Amino acid sequence of human nerve growth factor receptor (Sequence I.D. No. 24).

7B. Amino acid sequence of human CD4 receptor (Sequence I.D. No. 25).

7C. The interaction of Fas-associated phosphatase-1 to the C-terminal of nerve growth factor receptor (NGFR)(p75).

7D. Amino acid sequence of human colorectal mutant cancer protein (Sequence I.D. No. 26).

7E. Amino acid sequence of protein kinase C, alpha type.

7F. Amino acid sequence of serotonin 2A receptor (Sequence I.D. No. 27).

7G. Amino acid sequence of serotonin 2B receptor (Sequence I.D. No. 28).

7H. Amino acid sequence of adenomatosis polyposis coli protein (Sequence I.D. No. 29).

FIG. 8. Representation of the structural characteristics of p75 NGFR (low-affinity nerve growth factor receptor).

FIG. 9. Comparison of the C-terminal ends of Fas and p75 NGFR.

FIG. 10. In vitro interaction of ³⁵S-labeled FAP-1 with various receptors expressed as GST fusion proteins. The indicated GST fusion proteins immobilized on glutathione-Sepharose beads were incubated with in vitro translated, ³⁵S-labeled FAP-1 protein. After the beads were washed, retained FAP-1 protein was analyzed by SDS-PAGE and autoradiography.

FIGS. 11A and 11B. In vitro interaction ³⁵S-labeled FAP-1 with GST-p75 deletion mutants.

11A. Schematic representation of the GST fusion proteins containing the cytoplasmic domains of p75 and p75 deletion mutants. Binding of FAP-1 to the GST fusion proteins with various p75 deletion mutants is depicted at the right and is based on data from (11B).

11B. Interaction of in vitro translated, ³⁵S-labeled FAP-1 protein with various GST fusion proteins immobilized on glutathione-Sepharose beads. After the beads were washed, retained FAP-1 protein was analyzed by SDS-PAGE and autoradiography.

FIG. 12. The association between LexA-C-terminal cytoplasmic region of p75NGFR and VP16-FAP-1. The indicated yeast strains were constructed by transformation and the growth of colonies was tested. +/− indicates the growth of colonies on his ⁻ plate.

### DETAILED DESCRIPTION OF THE INVENTION

As used herein, amino acid residues are abbreviated as follows: A, Ala; C, Cys; D, Asp; E, Glu; F, Phe; G, Gly; H, His; I, Ile; K, Lys; L, Leu; M, Met; N, Asn; P, Pro; Q, Gln; R, Arg; S, Ser; T, Thr; V, Val; W, Trp; and Y, Tyr.

In order to facilitate an understanding of the material which follows, certain frequently occurring methods and/or terms are best described in Sambrook, et al., 1989.

The present invention provides for a composition capable of inhibiting specific binding between a signal-transducing protein and a cytoplasmic protein containing the amino acid sequence (G/S/A/E)-L-G-(F/I/L), wherein each—represents a peptide bond, each parenthesis encloses amino acids which are alternatives to one other, and each slash within such parentheses separating the alternative amino acids. Further, the cytoplasmic protein may contain the amino acid sequence (K/R/Q)-Xₙ-(G/S/A/E)-L-G-(F/I/L), wherein X represents any amino acid which is selected from the group comprising the twenty naturally occurring amino acids and n represents at least 2, but not more than 4. Specifically, in a preferred embodiment, the cytoplasmic protein contains the amino acid sequence SLGI.

The amino acid sequence (K/R/Q)-Xₙ-(G/S/A/E)-L-G-(F/I/L) is also well-known in the art as “GLGF (PDZ/DHR) amino acid domain.” As used herein, “GLGF (PDZ/DHR) amino acid domain” means the amino acid sequence (K/R/Q)-Xₙ-(G/S/A/E)-L-G-(F/I/L).

In a preferred embodiment, the signal-transducing protein has at its carboxyl terminus the amino acid sequence (S/T)-X-(V/I/L), wherein each—represents a peptide bond, each parenthesis encloses amino acids which are alternatives to one other, each slash within such parentheses separating the alternative amino acids, and the X represents any amino acid which is selected from the group comprising the twenty naturally occurring amino acids.

The compositions of the subject invention may be, but not limited to, antibodies, inorganic compounds, organic compounds, peptides, peptidomimetic compounds, polypeptides or proteins, fragments or derivatives which share some or all properties, e.g. fusion proteins. The composition may be naturally occurring and obtained by purification, or may be non-naturally occurring and obtained by synthesis.

Specifically, the composition may be a peptide containing the sequence (S/T)-X-(V/I/L)-COOH, wherein each—represents a peptide bond, each parenthesis encloses amino acids which are alternatives to one other, each slash within such parentheses separating the alternative amino acids, the X represents any amino acid which is selected from the group comprising the twenty naturally occurring amino acids. In preferred embodiments, the peptide contains one of the following sequences: DSENSNFRNEIQSLV, RNEIQSLV, NEIQSLV, EIQSLV, IQSLV, QSLV, SLV, IPPDSEDGNEEQSLV, DSEMYNFRSQLASVV, IDLASEFLFLSNSFL, PPTCSQANSGRISTL, SDSNMNMNELSEV, QNFRTYIVSFV, RETIESTV, RGFISSLV, TIQSVI, ESLV. A further preferred embodiment would be an organic compound which has the sequence Ac-SLV-COOH, wherein the Ac represents an acetyl and each—represents a peptide bond.

An example of the subject invention is provided infra. Acetylated peptides may be automatically synthesized on an Advanced ChemTech ACT357 using previously published procedures by analogy. Wang resin was used for each run and N^{α}-Fmoc protection was used for all amino acids, and then 20% piperidine/DMF and coupling was completed using DIC/HOBt and subsequently HBTU/DIEA. After the last amino acid was coupled, the growing peptide on the resin was acetylated with Ac₂O/DMF. The acetylated peptide was purified by HPLC and characterized by FAB-MS and ¹H-NMR.

Further, one skilled in the art would know how to construct derivatives of the above-described synthetic peptides coupled to non-acetyl groups, such as amines.

This invention also provides for a composition capable of inhibiting specific binding between a signal-transducing protein having at its carboxyl terminus the amino acid sequence (S/T)-X-(V/I/L), wherein each—represents a peptide bond, each parenthesis encloses amino acids which are alternatives to one other, each slash within such parentheses separating the alternative amino acids, the X represents any amino acid which is selected from the group comprising the twenty naturally occurring amino acids, and a cytoplasmic protein.

The compositions of the subject invention includes antibodies, inorganic compounds, organic compounds, peptides, peptidomimetic compounds, polypeptides or proteins, fragments or derivatives which share some or all properties, e.g. fusion proteins.

This invention also provides a method of identifying a compound capable of inhibiting specific binding between a signal-transducing protein and a cytoplasmic protein containing the amino acid sequence (G/S/A/E)-L-G-(F/I/L), wherein each—represents a peptide bond, each parenthesis encloses amino acids which are alternatives to one other, each slash within such parentheses separating the alternative amino acids, which comprises (a) contacting the cytoplasmic protein bound to the signal-transducing protein with a plurality of compounds under conditions permitting binding between a known compound previously shown to be able to displace the signal-transducing protein bound to the cytoplasmic protein and the bound cytoplasmic protein to form a complex; and (b) detecting the displaced signal-transducing protein or the complex formed in step (a) wherein the displacement indicates that the compound is capable of inhibiting specific binding between the signal-transducing protein and the cytoplasmic protein.

The inhibition of the specific binding between the signal-transducing protein and the cytoplasmic protein may affect the transcription activity of a reporter gene.

Further, in step (b), the displaced cytoplasmic protein or the complex is detected by comparing the transcription activity of a reporter gene before and after the contacting with the compound in step (a), where a change of the activity indicates that the specific binding between the signal-transducing protein and the cytoplasmic protein is inhibited and the signal-transducing protein is displaced.

As used herein, the “transcription activity of a reporter gene” means that the expression level of the reporter gene will be altered from the level observed when the signal-transducing protein and the cytoplasmic protein are bound. One can also identify the compound by detecting other biological functions dependent on the binding between the signal-transducing protein and the cytoplasmic protein. Examples of reporter genes are numerous and well-known in the art, including, but not limited to, histidine resistant genes, ampicillin resistant genes, β-galactosidase gene.

Further the cytoplasmic protein may be bound to a solid support. Also the compound may be bound to a solid support and comprises an antibody, an inorganic compound, an organic compound, a peptide, a peptidomimetic compound, a polypeptide or a protein.

An example of the method is provided infra. One can identify a compound capable of inhibiting specific binding between the signal-transducing protein and the cytoplasmic protein using direct methods of detection such as immuno-precipitation of the cytoplasmic protein and the compound bound to a detectable marker. Further, one could use indirect methods of detection that would detect the increase or decrease in levels of gene expression. As discussed infra, one could construct synthetic peptides fused to a LexA DNA binding domain. These constructs would be transformed into the L40-strain with an appropriate cell line having an appropriate reporter gene. One could then detect whether inhibition had occurred by detecting the levels of expression of the reporter gene. In order to detect the expression levels of the reporter gene, one skilled in the art could employ a variety of well-known methods, e.g. two-hybrid systems in yeast, mammals or other cells.

Further, the contacting of step (a) may be in vitro, in vivo, and specifically in an appropriate cell, e.g. yeast cell or mammalian cell. Examples of mammalian cells include, but not limited to, the mouse fibroblast cell NIH 3T3, CHO cells, HeLa cells, Ltk⁻ cells, Cos cells, etc.

Other suitable cells include, but are not limited to, prokaryotic or eukaryotic cells, e.g. bacterial cells (including gram positive cells), fungal cells, insect cells, and other animals cells.

Further, the signal-transducing protein may be a cell surface receptor, signal transducer protein, or a tumor suppressor protein. Specifically, the cell surface protein is the Fas receptor and may be expressed in cells derived from organs including, but not limited to, thymus, liver, kidney, colon, ovary, breast, testis, spleen, lung, stomach, prostate, uterus, skin, head, and neck, or expressed in cells comprising T-cells and B-cells. In a preferred embodiment, the T-cells are Jurkat T-cells.

Further, the cell-surface receptor may be a CD4 receptor, p75 receptor, serotonin 2A receptor, or serotonin 2B receptor.

Further, the signal transducer protein may be Protein Kinase-C-α-type.

Further, the tumor suppressor protein may be a adenomatosis polyposis coli tumor suppressor protein or colorectal mutant cancer protein.

Further, the cytoplasmic protein contains the amino acid sequence SLGI, specifically Fas-associated phosphatase-1.

This invention also provides a method of identifying a compound capable of inhibiting specific binding between a signal-transducing protein having at its carboxyl terminus the amino acid sequence (S/T)-X-(V/I/L), wherein each—represents a peptide bond, each parenthesis encloses amino acids which are alternatives to one other, each slash within such parentheses separating the alternative amino acids, the X represents any amino acid which is selected from the group comprising the twenty naturally occurring amino acids, and a cytoplasmic protein which comprises (a) contacting the signal-transducing protein bound to the cytoplasmic protein with a plurality of compounds under conditions permitting binding between a known compound previously shown to be able to displace the cytoplasmic protein bound to the signal-transducing protein and bound signal-transducing protein to form a complex; and (b) detecting the displaced cytoplasmic protein or the complex of step (a), wherein the displacement indicates that the compound is capable of inhibiting specific binding between the signal-transducing protein and the cytoplasmic protein. The inhibition of the specific binding between the signal-transducing protein and the cytoplasmic protein affects the transcription activity of a reporter gene. Further, in step (b), the displaced signal-transducing protein or the complex is detected by comparing the transcription activity of a reporter gene before and after the contacting with the compound in step (a), where a change of the activity indicates that the specific binding between the signal-transducing protein and the cytoplasmic protein is inhibited and the cytoplasmic protein is displaced.

Further, in step (b), the displaced cytoplasmic protein or the complex is detected by comparing the transcription activity of a reporter gene before and after the contacting with the compound in step (a), where a change of the activity indicates that the specific binding between the signal-transducing protein and the cytoplasmic protein is inhibited and the signal-transducing protein is displaced.

As used herein, the “transcription activity of a reporter gene” means that the expression level of the reporter gene will be altered from the level observed when the signal-transducing protein and the cytoplasmic protein are bound. One can also identify the compound by detecting other biological functions dependent on the binding between the signal-transducing protein and the cytoplasmic protein. Examples of reporter genes are numerous and well-known in the art, including, but not limited to, histidine resistant genes, ampicillin resistant genes, β-galactosidase gene.

Further, the cytoplasmic protein may be bound to a solid support or the compound may be bound to a solid support, comprises an antibody, an inorganic compound, an organic compound, a peptide, a peptidomimetic compound, a polypeptide or a protein.

An example of the method is provided infra. One could identify a compound capable of inhibiting specific binding between the signal-transducing protein and the cytoplasmic protein using direct methods of detection such as immuno-precipitation of the cytoplasmic protein and the compound bound with a detectable marker. Further, one could use indirect methods of detection that would detect the increase or decrease in levels of gene expression. As discussed infra, one could construct synthetic peptides fused to a LexA DNA binding domain. These constructs would be transformed into L40-strain with an appropriate cell line having a reporter gene. One could then detect whether inhibition had occurred by detecting the levels of the reporter gene. Different methods are also well known in the art, such as employing a yeast two-hybrid system to detect the expression of a reporter gene.

Further the contacting of step (a) can be in vitro or in vivo, specifically in a yeast cell or a mammalian cell. Examples of mammalian cells include, but not limited to, the mouse fibroblast cell NIH 3T3, CHO cells, HeLa cells, Ltk⁻ cells, Cos cells, etc.

Other suitable cells include, but are not limited to, prokaryotic or eukaryotic cells, e.g. bacterial cells (including gram positive cells), fungal cells, insect cells, and other animals cells.

Further, the signal-transducing protein is a cell surface receptor, signal transducer protein, or a tumor suppressor protein. Specifically, the cell surface protein is the Fas receptor and is expressed in cells derived from organs comprising thymus, liver, kidney, colon, ovary, breast, testis, spleen, stomach, prostate, uterus, skin, head and neck, or expressed in cells comprising T-cells and B-cells. In a preferred embodiment, the T-cells are Jurkat T-cells.

Further, the cell-surface receptor may be a CD4 receptor, p75 receptor, serotonin 2A receptor, or serotonin 2B receptor.

Further, the signal transducer protein may be Protein Kinase-C-α-type.

Further, the tumor suppressor protein may be a adenomatosis polyposis coli tumor suppressor protein or colorectal mutant cancer protein.

Further, the cytoplasmic protein contains the amino acid sequence SLGI, specifically Fas-associated phosphatase-1.

This invention also provides a method of inhibiting the proliferation of cancer cells comprising the above-described composition, specifically, wherein the cancer cells are derived from organs including, but not limited to, thymus, liver, kidney, colon, ovary, breast, testis, spleen, stomach, prostate, uterus, skin, head and neck, or wherein the cancer cells are derived from cells comprising T-cells and B-cells.

This invention also provides a method of inhibiting the proliferation of cancer cells comprising the compound identified by the above-described method, wherein the cancer cells are derived from organs including, but not limited to, thymus, liver, kidney, colon, ovary, breast, testis, spleen, stomach, prostate, uterus, skin, head and neck, or wherein the cancer cells are derived from cells comprising T-cells and B-cells.

The invention also provides a method of treating cancer in a subject which comprises introducing to the subject's cancerous cells an amount of the above-described composition effective to result in apoptosis of the cells, wherein the cancer cells are derived from organs including, but not limited to, thymus, liver, kidney, colon, ovary, breast, testis, spleen, stomach, prostate, uterus, skin, head and neck, or wherein the cancer cells are derived from cells comprising T-cells and B-cells.

As used herein “apoptosis” means programmed cell death of the cell. The mechanisms and effects of programmed cell death differs from cell lysis. Some observable effects of apoptosis are: DNA fragmentation and disintegration into small membrane-bound fragments called apoptotic bodies.

Means of detecting whether the composition has been effective to result in apoptosis of the cells are well-known in the art. One means is by assessing the morphological change of chromatin using either phase contrast or fluorescence microscopy.

The invention also provides for a method of inhibiting the proliferation of virally infected cells comprising the above-described composition or the compound identified by the above-described, wherein the virally infected cells comprise Hepatitis B virus, Epstein-Barr virus, influenza virus, Papilloma virus, Adeno virus, Human T-cell lymphtropic virus, type 1 or HIV.

The invention also provides a method of treating a virally-infected subject which comprises introducing to the subject's virally-infected cells the above-described composition effective to result in apoptosis of the cells or the compound identified by the above-described method of claim 27 effective to result in apoptosis of the cells, wherein the virally infected cells comprise the Hepatitis B virus, Epstein-Barr virus, influenza virus, Papilloma virus, Adeno virus, Human T-cell lymphtropic virus, type 1 or HIV.

Means of detecting whether the composition has been effective to result in apoptosis of the cells are well-known in the art. One means is by assessing the morphological change of chromatin using either phase contrast or fluorescence microscopy.

This invention also provides for a pharmaceutical composition comprising the above-described composition of in an effective amount and a pharmaceutically acceptable carrier.

This invention also provides for a pharmaceutical composition comprising the compound identified by the above-described method of in an effective amount and a pharmaceutically acceptable carrier.

This invention further provides a composition capable of specifically binding a signal-transducing protein having at its carboxyl terminus the amino acid sequence (S/T)-X-(V/L/I), wherein each—represents a peptide bond, each parenthesis encloses amino acids which are alternatives to one other, each slash within such parentheses separating the alternative amino acids, and the X represents any amino acid which is selected from the group comprising the twenty naturally occurring amino acids. The composition may contain the amino acid sequence (G/S/A/E)-L-G-(F/I/L), wherein each—represents a peptide bond, each parenthesis encloses amino acids which are alternatives to one other, and each slash within such parentheses separating the alternative amino acids. In a preferred embodiment, the composition contains the amino acid sequence (K/R/Q)-Xₙ-(G/S/A/E)-L-G-(F/I/L) wherein X represents any amino acid which is selected from the group comprising the twenty naturally occurring amino acids and n represents at least 2, but not more than 4. In another preferred embodiment, the composition contains the amino acid sequence SLGI.

This invention further provides a method for identifying compounds capable of binding to a signal-transducing protein having at its carboxyl terminus the amino acid sequence (S/T)-X-(V/L/I), wherein each—represents a peptide bond, each parenthesis encloses amino acids which are alternatives to one other, each slash within such parentheses separating the alternative amino acids, the X represents any amino acid which is selected from the group comprising the twenty naturally occurring amino acids, which comprises (a) contacting the signal-transducing protein with a plurality of compounds under conditions permitting binding between a known compound previously shown to be able to bind to the signal-transducing protein to form a complex; and (b) detecting the complex formed in step (a) so as to identify a compound capable of binding to the signal-transducing protein. Specifically, the identified compound contains the amino acid sequence (G/S/A/E)-L-G-(F/I/L). In a further preferred embodiment, the identified compound contains the amino acid sequence SLGI.

Further, in the above-described method, the signal-transducing protein may be bound to a solid support. Also, the compound may be bound to a solid support, and may comprise an antibody, an inorganic compound, an organic compound, a peptide, a peptidomimetic compound, a polypeptide or a protein.

Further, the signal-transducing protein may be a cell-surface receptor or a signal transducer. Specifically, the signal-transducing protein may be the Fas receptor, CD4 receptor, p75 receptor, serotonin 2A receptor, serotonin 2B receptor, or protein kinase-C-α-type.

This invention also provides a method of restoring negative regulation of apoptosis in a cell comprising the above-described composition or a compound identified by the above-described method.

As used herein “restoring negative regulation of apoptosis” means enabling the cell from proceeding onto programmed cell death.

For example, cells that have functional Fas receptors and Fas-associated phosphatase 1 do not proceed onto programmed cell death or apoptosis due to the negative regulation of Pas by the phosphatase. However, if Fas-associated phosphatase 1 is unable to bind to the carboxyl terminus of the Fas receptor ((S/T)-X-(V/L/I) region), e.g. mutation or deletion of at least one of the amino acids in the amino acid sequence (G/S/A/E)-L-G-(F/I/L), the cell will proceed to apoptosis. By introducing a compound capable of binding to the carboxyl terminus of the Fas receptor, one could mimic the effects of a functional phosphatase and thus restore the negative

regulation of apoptosis. This invention also provides a method of preventing apoptosis in a cell comprising the above-described composition or a compound identified by the above-described method.

This invention also provides a means of treating pathogenic conditions caused by apoptosis of relevant cells comprising the above-described composition or the compound identified by the above-described method.

This invention is illustrated in the Experimental Details section which follows. These sections are set forth to aid in an understanding of the invention but are not intended to, and should not be construed to, limit in any way the invention as set forth in the claims which follow thereafter.

First Series of Experiments

Experimental Details

Methods and Materials

1. Screening a Semi-Random and Random Peptide Library.

To create numerous mutations in a restricted DNA sequence, PCR mutagenesis with degenerate oligonucleotides was employed according to a protocol described elsewhere (Hill, et al. 1987). Based on the homology between human and rat, two palindromic sequences were designed for construction of semi-random library. The two primers used were 5′-CGGAATTCNNNNNNNNNAACAGCNNNNNNNNNAATGAANNNCAAAGTCTGNN NTGAGGATCCTCA-3′ (Seq. I.D. No. 30) and 5′-CGGAATTCGACTCAGAANNNNNNAACTTCAGANNNNNNATCNNNNNNNNNGT CTGAGGATCCTCA-3′ (Seq. I.D. No. 31). Briefly, the two primers (each 200 pmol), purified by HPLC, were annealed at 70° C. for 5 minutes and cooled at 23° C. for 60 minutes. A Klenow fragment (5 U) was used for filling in with a dNTP mix (final concentration, 1 mM per each dNTP) at 23° C. for 60 minutes. The reaction was stopped with 1 μl of 0.5 M EDTA and the DNA was purified with ethanol precipitation. The resulting double-stranded DNA was digested with EcoRI and BamHI and re-purified by electrophoresis on non-denaturing polyacrylamide gels. The double-strand oligonucleotides were then ligated into the EcoRI-BamHI sites of the pBTM116 plasmid. The ligation mixtures were electroporated into the E. coli XL1-Blue MRF′ (Stratagene) for the plasmid library. The large scale transformation was carried out as previously reported. The plasmid library was transformed into L40-strain cells (MATa, trp1, leu2, his3, ade2, LYS2: (lexAop)⁴-HIS3, URA3::(lexAop)⁸-lacZ) carrying the plasmid pVP16-31 containing a FAP-1 cDNA (Sato, et al. 1995). Clones that formed on histidine-deficient medium (His⁺) were transferred to plates containing 40 μg/ml X-gal to test for a blue reaction product (β-gal⁺) in plate and filter assays. The clones selected by His⁺ and β-gal⁺ assay were tested for further analysis. The palindromic oligonucleotide, 5′-CGGAATTC-(NNN)₄₋₁₅-TGAGGATCCTCA-3′ (Seq. I.D. No. 32), was used for the construction of the random peptide library.

2. Synthesis of Peptides

Peptides were automatically synthesized on an Advanced ChemTech ACT357 by analogy to published procedures (Schnorrenberg and Gerhardt, 1989). Wang resin (0.2-0.3 mmole scale) was used for each run and N′-Fmoc protection was employed for all amino acids. Deprotection was achieved by treatment with 20% piperidine/DMF and coupling was completed using DIC/HOBt and subsequent HBTU/DIEA. After the last amino acid was coupled, the growing peptide on the resin was acetylated with Ac₂O/DMF. The peptide was cleaved from the resin with concomitant removal of all protecting groups by treating with TFA. The acetylated peptide was purified by HPLC and characterized by FAB-MS and ¹H-NMR.

3. Inhibition Asssay of Fas/FAP-1 Binding Using the C-Terminal 15 Amino Acids of Fas.

HFAP-10 cDNA (Sato, et al. 1995) subcloned into the Bluescript vector pSK-II (Stratagene) was in vitro-translated from an internal methionine codon in the presence of ³⁵S-L-methionine using a coupled in vitro transcription/translation system (Promega, TNT lysate) and T7 RNA polymerase. The resulting ³⁵S-labeled protein was incubated with GST-Fas fusion proteins that had been immobilized on GST-Sepharose 4B affinity beads (Pharmacia) in a buffer containing 150 mM NaCl, 50 mM Tris [pH 8.0], 5 mM DTT, 2 mM EDTA, 0.1% NP-40, 1 mM PMSF, 50 μg/ml leupeptin, 1 mM Benzamidine, and 7 μg/ml pepstatin for 16 hours at 4° C. After washing vigorously 4 times in the same buffer, associated proteins were recovered with the glutathione-Sepharose beads by centrifugation, eluted into boiling Laemmli buffer, and analyzed by SDS-PAGE and fluorography.

4. Inhibition Assay of Terminal 15 Amino Acids of Fas and Inhibitory Effect of Fas/FAP-1 Binding Using Diverse Tripeptides.

In vitro-translated [³⁵]HFAP-1 was purified with a NAP-5 column (Pharmacia) and incubated with 3 μM of GST-fusion proteins for 16 hours at 4° C. After washing 4 times in the binding buffer, radioactivity incorporation was determined in a b counter. The percentage of binding inhibition was calculated as follows: percent inhibition=[radioactivity incorporation using GST-Fas (191-335) with peptides−radioactivity incorporation using GST-Fas (191-320) with peptides]/[radioactivity incorporation using GST-Fas (191-335) without peptides−radioactivity incorporation using GST-Fas (191-320) without peptides]. n=3.

5. Interaction of the C-Terminal 3 Amino Acids of Fas with FAP-1 in Yeast and in Vitro.

The bait plasmids, pBTM116 (LexA)-SLV, -PLV, -SLY, and -SLA, were constructed and transformed into L40-strain with pVP16-FAP-l or -ras. Six independent clones from each transformants were picked up for the analysis of growth on histidine-deficient medium. GST-Fas, -SLV, and PLV were purified with GST-Sepharose 4B affinity beads (Pharmacia). The methods for in vitro binding are described above.

6. Immuno-Precipitation of Native Fas with GST-FAP-1 and Inhibition of Fas/FAP-1 Binding with Ac-SLV.

GST-fusion proteins with or without FAP-1 were incubated with cell extracts from Jurkat T-cells expressing Fas. The bound Fas was detected by Western analysis using anti-Fas monoclonal antibody (F22120, Transduction Laboratories). The tripeptides, Ac-SLV and Ac-SLY were used for the inhibition assay of Fas/FAP-1 binding.

7. Microinjection of Ac-SLV into the DLD-1 Cell Line.

DLD-1 human colon cancer cells were cultured in RPMI 1640 medium containing 10% FCS. For microinjection, cells were plated on CELLocate (Eppendorf) at 1×10⁵ cells/2 ml in a 35 mm plastic culture dish and grown for 1 day. Just before microinjection, Fas monoclonal antibodies CH11 (MBL International) was added at the concentration of 500 ng/ml. All microinjection experiments were performed using an automatic microinjection system (Eppendorf transjector 5246, micro-manipulator 5171 and Femtotips) (Pantel, et al. 1995). Synthetic tripeptides were suspended in 0.1% (w/v) FITC-Dextran (Sigma)/K-PBS at the concentration of 100 mM. The samples were microinjected into the cytoplasmic region of DLD-1 cells. Sixteen to 20 hours postinjection, the cells were washed with PBS and stained with 10 μg/ml Hoechst 33342 in PBS. After incubation at 37° C. for 30 minutes, the cells were photographed and the cells showing condensed chromatin were counted as apoptotic.

8. Quantitation of Apoptosis in Microinjected DLD-1 Cells.

For each experiment, 25-100 cells were microinjected. Apoptosis of microinjected cells was determined by assessing morphological changes of chromatin using phase contrast and fluorescence microscopy (Wang, et al., 1995; McGahon, et al., 1995). The data are means +/−S.D. for two or three independent determinations.

Discussion

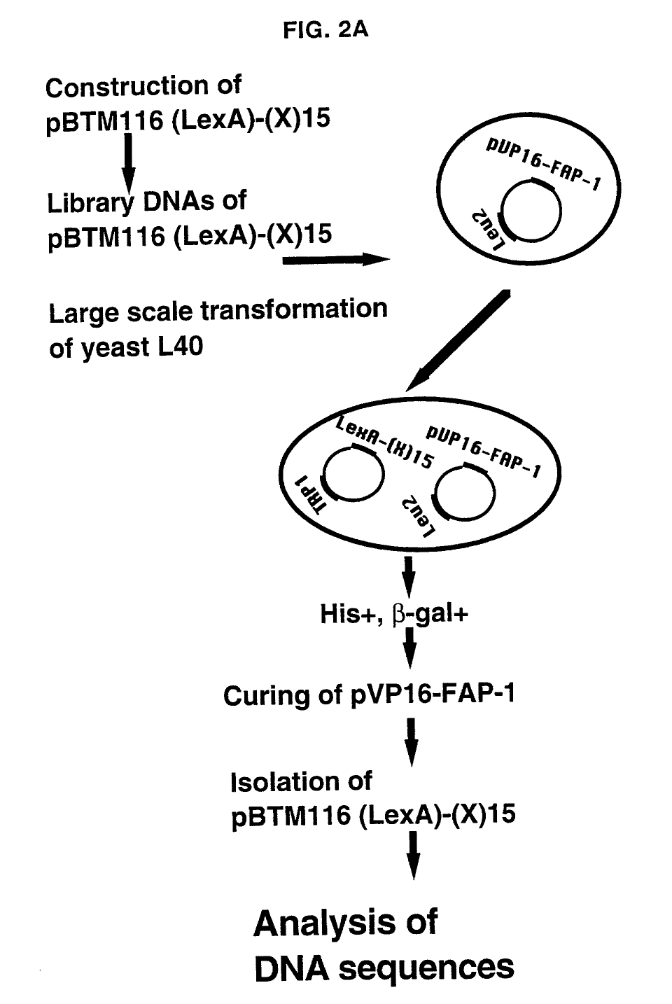

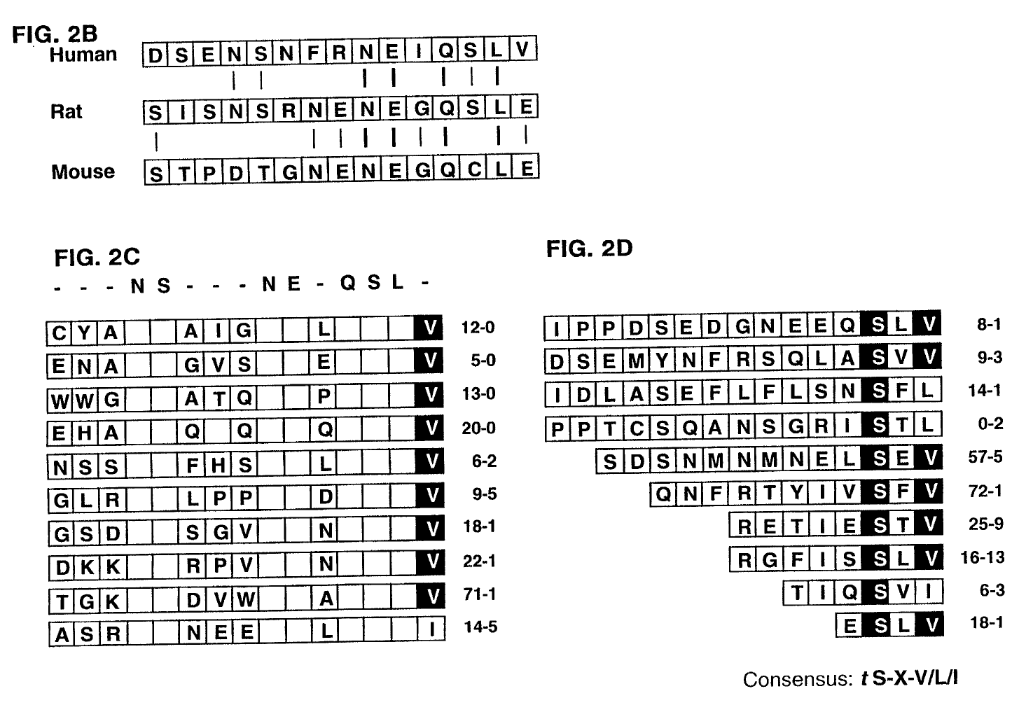

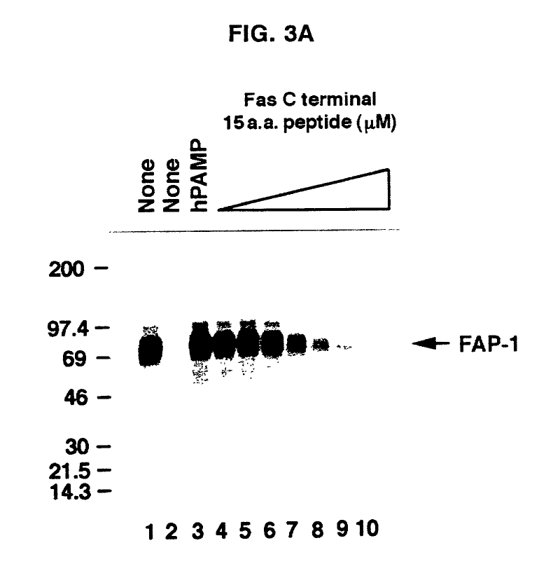

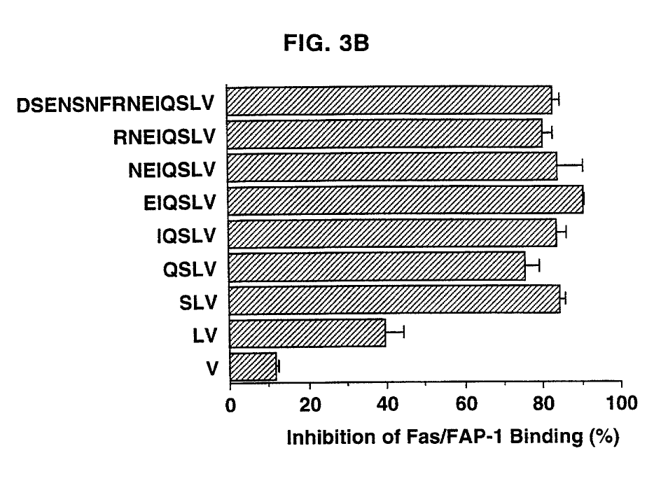

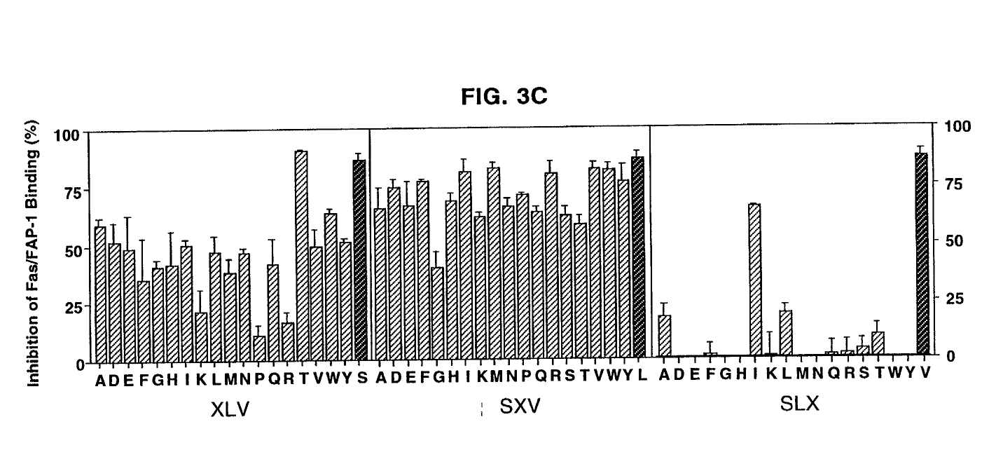

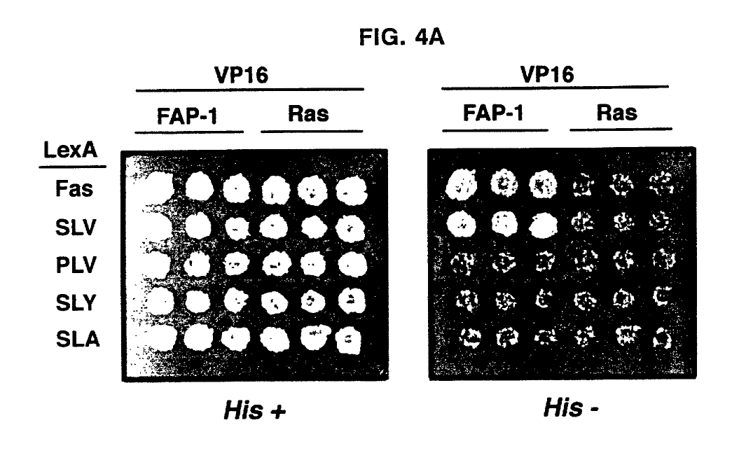

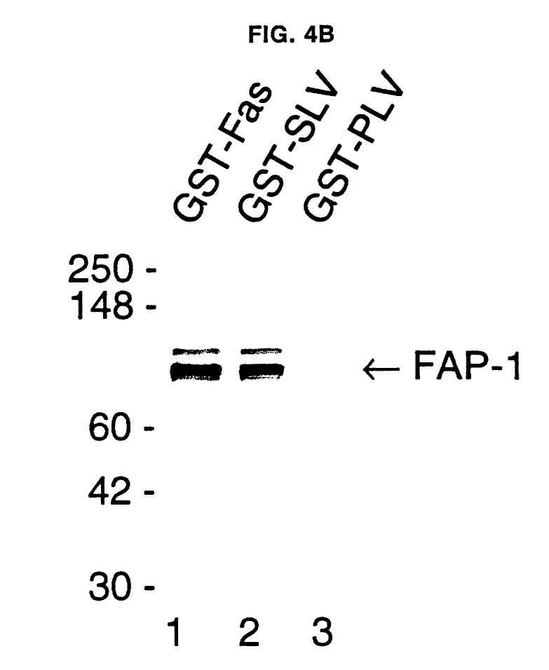

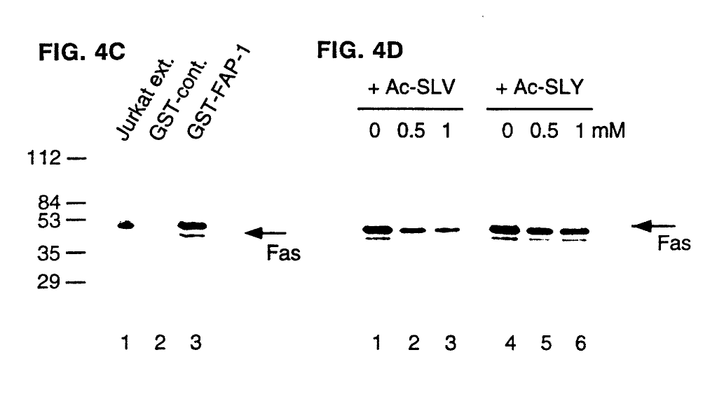

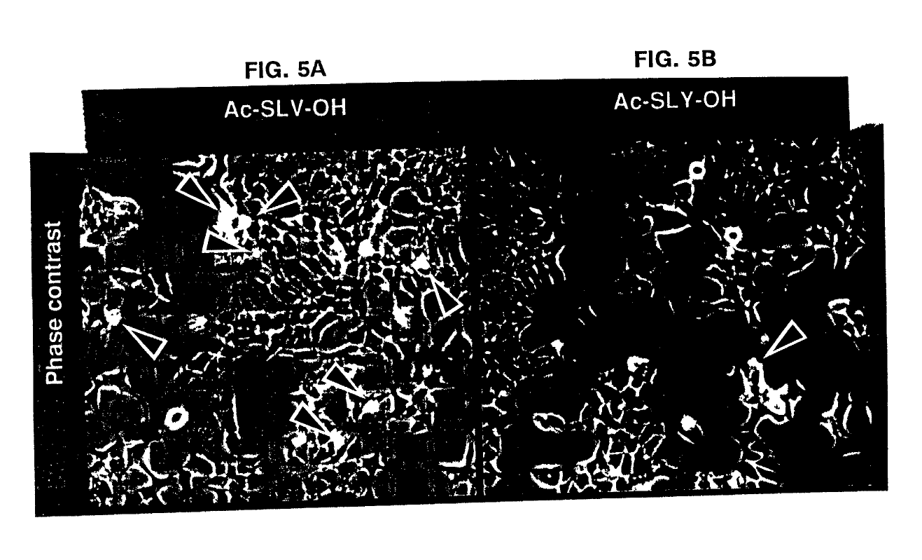

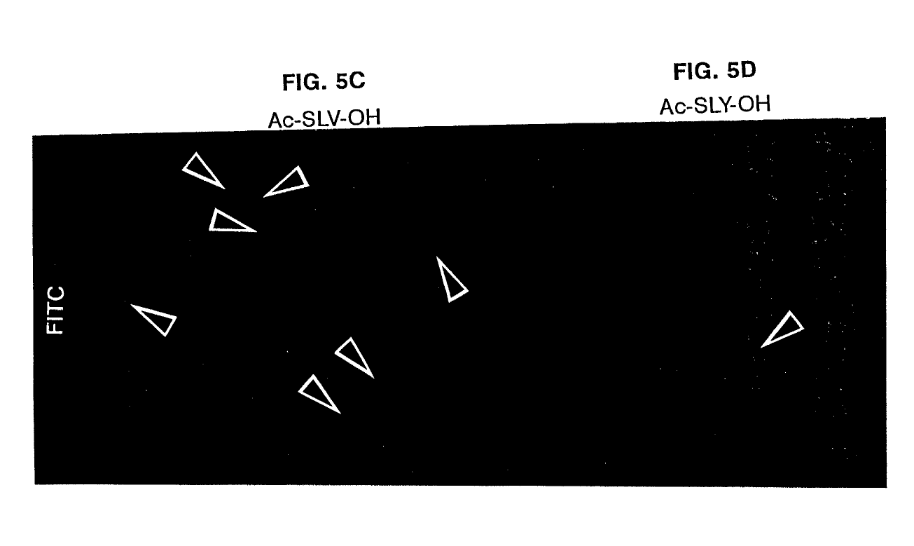

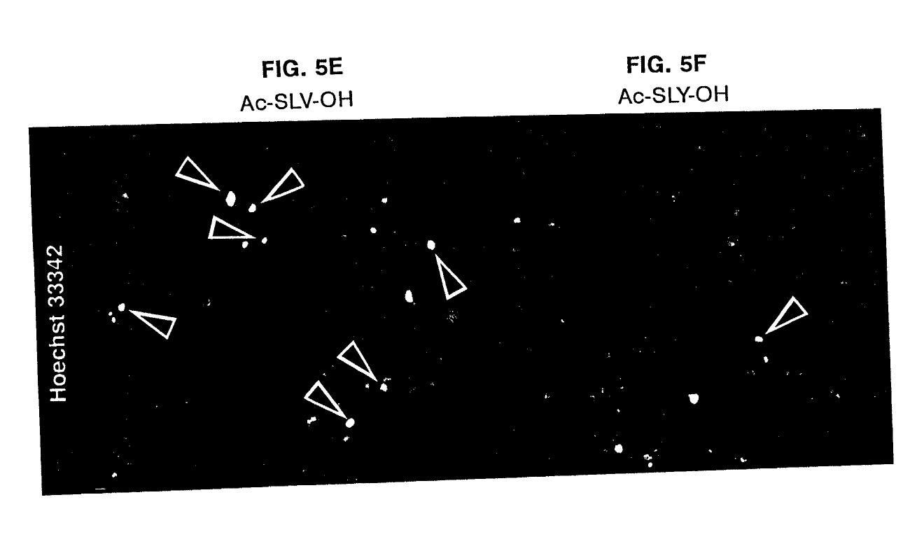

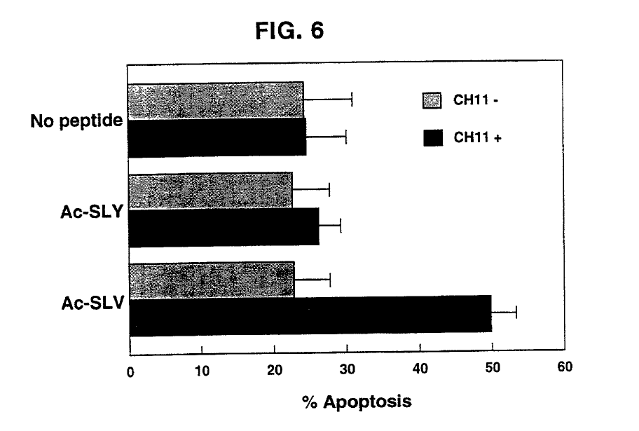

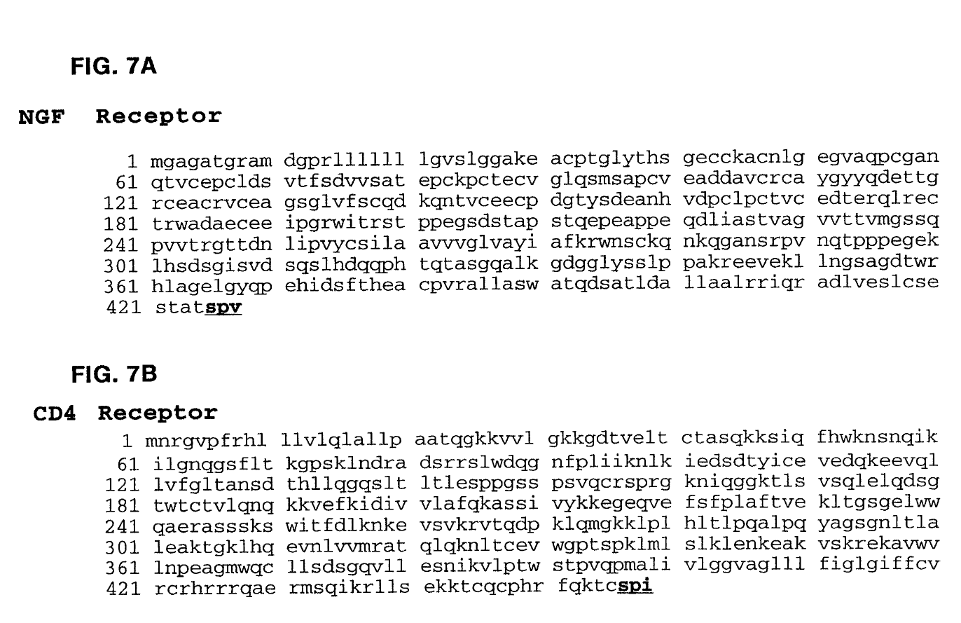

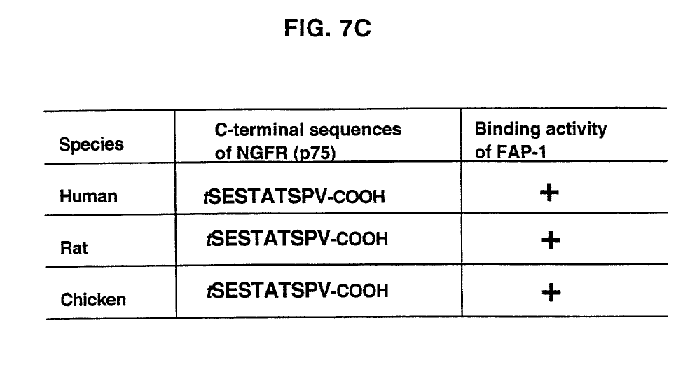

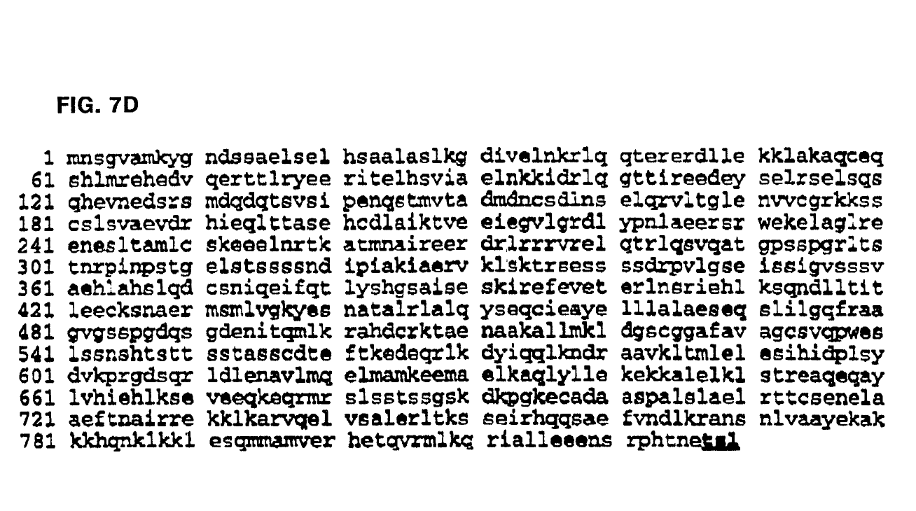

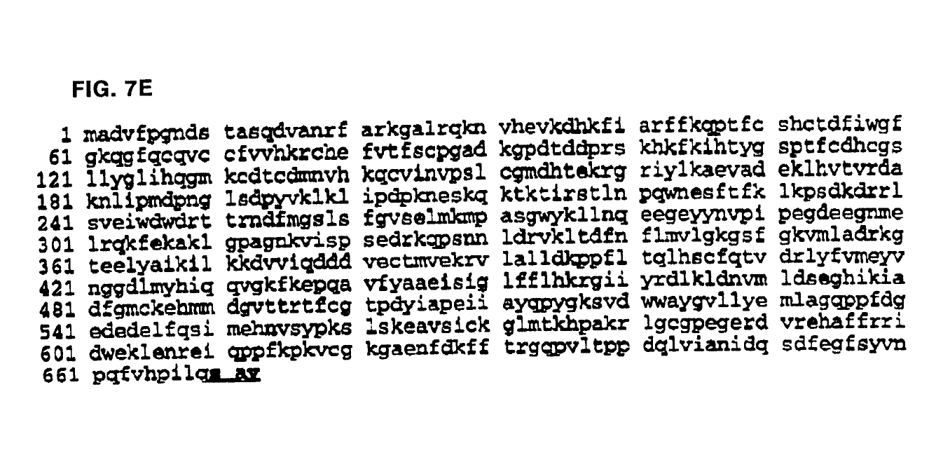

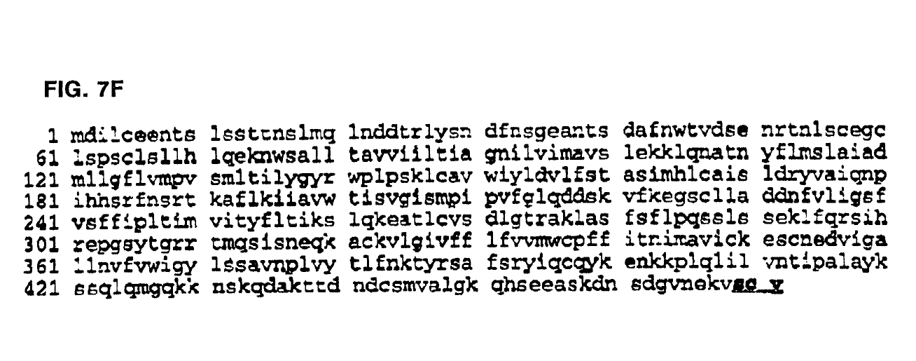

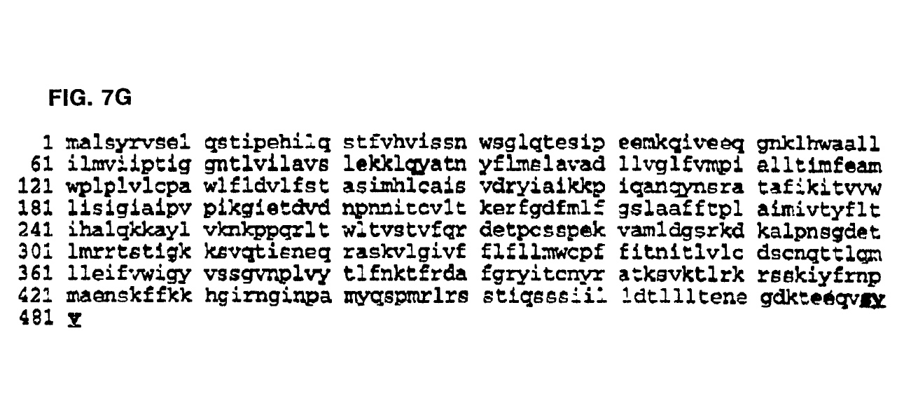

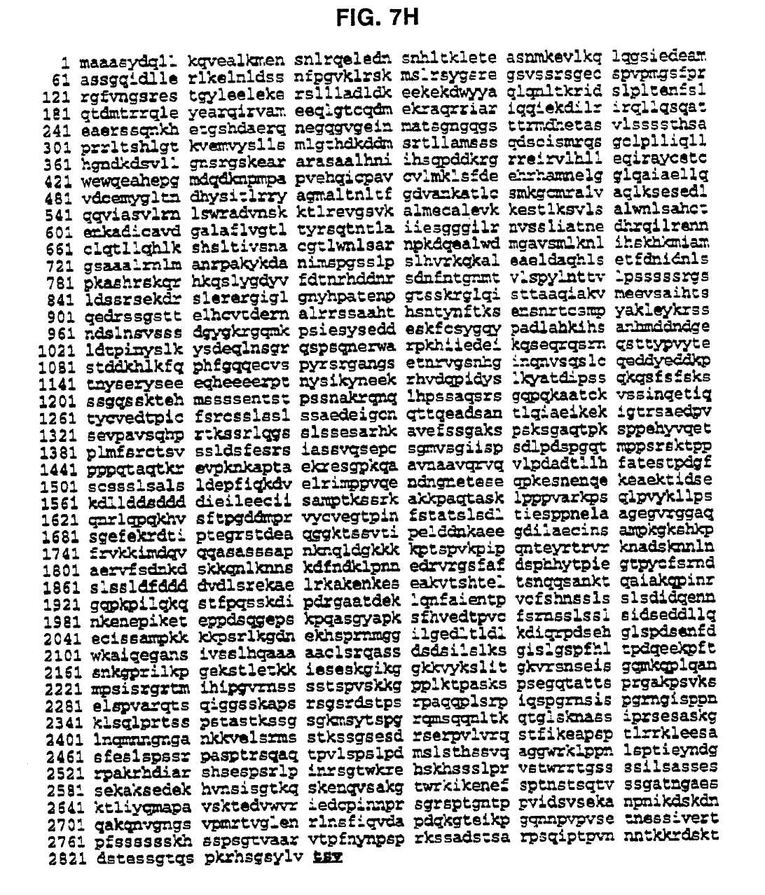

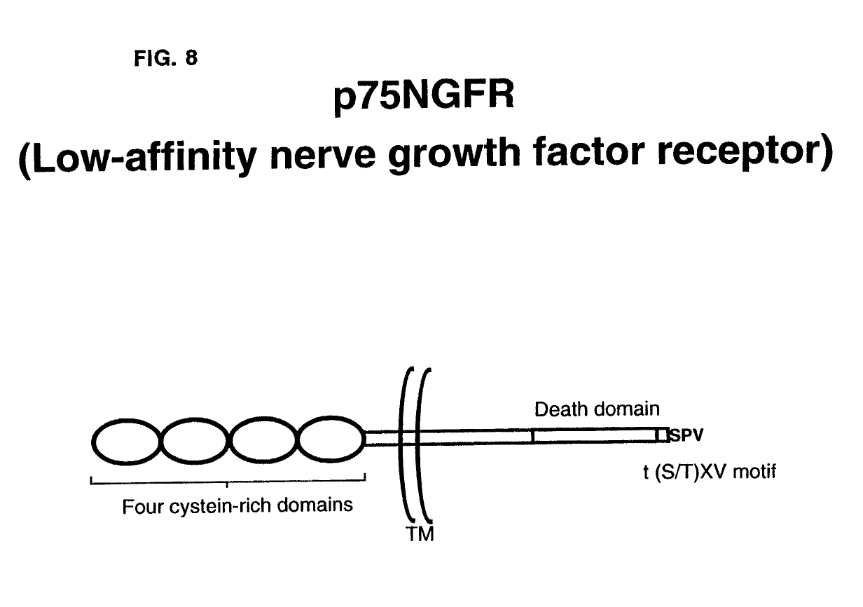

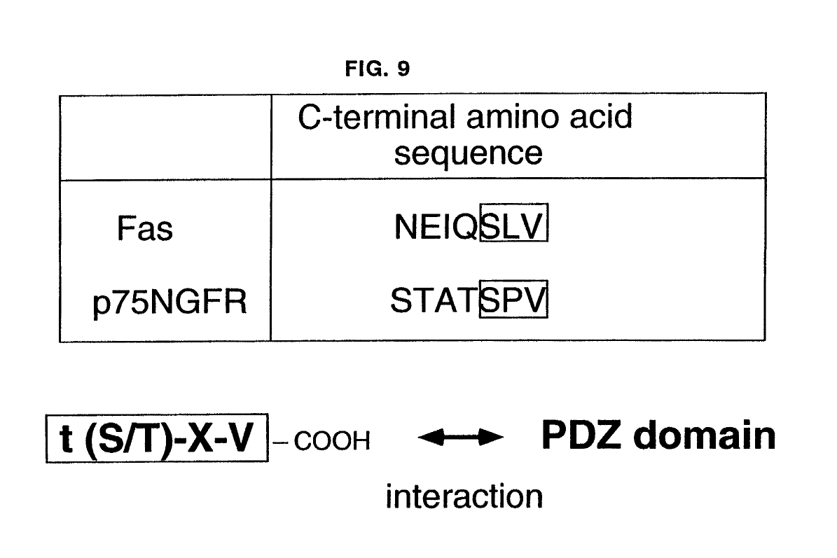

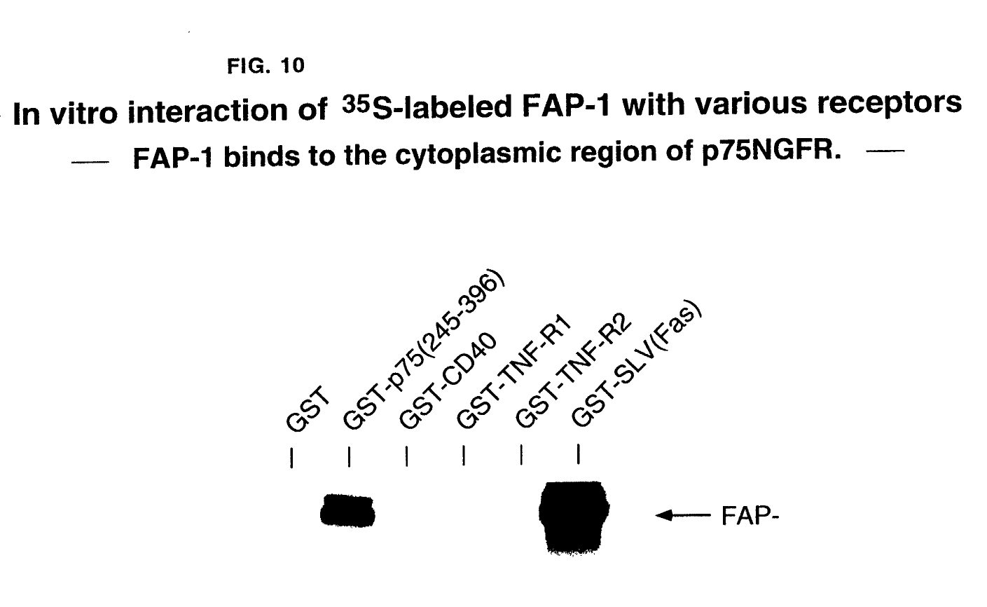

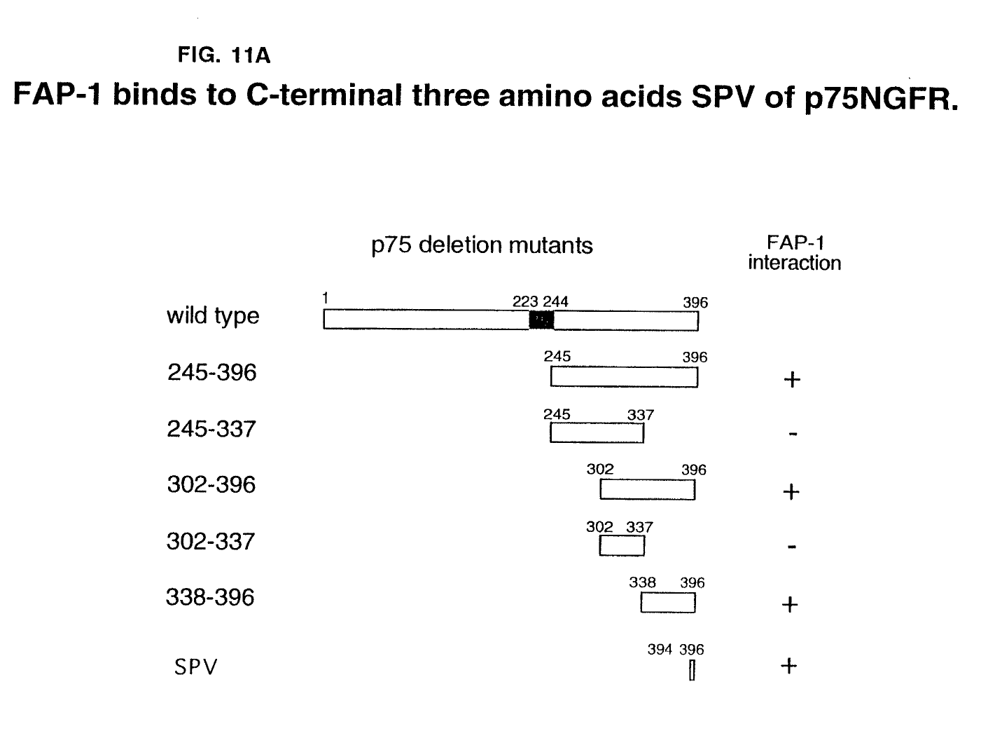

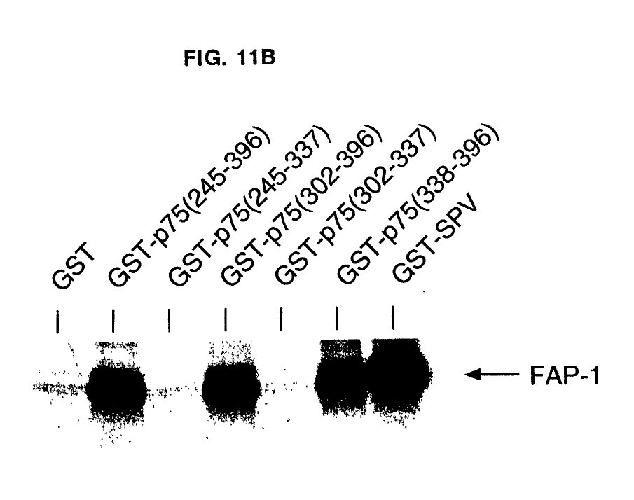

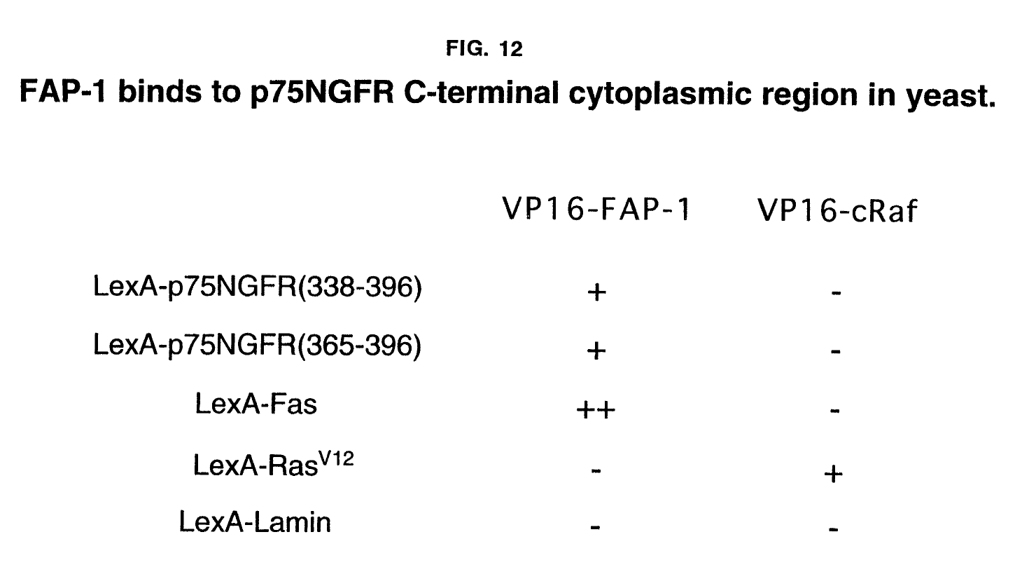

In order to identify the minimal peptide stretch in the C-terminal region of the Fas receptor necessary for FAP-1 binding, an in vitro inhibition assay of Fas/FAP-1 binding was used using a series of synthetic peptides as well as yeast two-hybrid system peptide libraries (FIG. 2A). First, semi-random libraries (based on the homology between human and rat Fas) (FIGS. 2B and 2C) of 15 amino acids fused to a LexA DNA binding domain were constructed and co-transformed into yeast strain L40 with pVP16-31 (Sato, et al. 1995) that was originally isolated as FAP-1. After the selection of 200 His⁺ colonies from an initial screen of 5.0×10⁶ (Johnson, et al. 1986) transformants, 100 colonies that were β-galactosidase positive were picked for further analysis. Sequence analysis of the library plasmids encoding the C-terminal 15 amino acids revealed that all of the C-termini were either valine, leucine or isoleucine residues. Second, a random library of 4-15 amino acids fused to a LexA DNA binding domain was constructed and screened according to this strategy (FIG. 2D). Surprisingly, all of the third amino acid residues from the C-termini were serine, and the results of C-terminal amino acid analyses were identical to the screening of the semi-random cDNA libraries. No other significant amino acid sequences were found in these library screenings, suggesting that the motifs of the last three amino acids (tS-X-V/L/I) are very important for the association with the third PDZ domain of FAP-1 and play a crucial role in protein-protein interaction as well as for the regulation of Fas-induced apoptosis. To further confirm whether the last three amino acids are necessary and sufficient for Fas/FAP-1 binding, plasmids of the LexA-SLV, -PLV, -PLY, -SLY, and -SLA fusion proteins were constructed and co-transformed into yeast with pVP16-FAP-1. The results showed that only LexA-SLV associated with FAP-1, whereas LexA-PLV, -PLY, -SLY, and -SLA did not (FIG. 4A). In vitro binding studies using various GST-tripeptide fusions and in vitro-translated FAP-1 were consistent with these results (FIG. 4B).

In addition to yeast two-hybrid approaches, in vitro inhibition assay of Fas/FAP-1 binding was also used. First, a synthetic peptide of the C-terminal 15 amino acids was tested whether it could inhibit the binding of Fas and FAP-1 in vitro (FIG. 3A). The binding of in vitro-translated FAP-1 to GST-Fas was dramatically reduced and dependent on the concentration of the synthetic 15 amino acids of Fas. In contrast with these results, human PAMP peptide (Kitamura, et al. 1994) as a negative control had no effect on Fas/FAP-1 binding activity under the same biochemical conditions. Second, the effect of truncated C-terminal synthetic peptides of Fas on Fas/FAP-1 binding in vitro was examined. As shown in FIG. 3B, only the three C-terminal amino acids (Ac-SLV) were sufficient to obtain the same level of inhibitory effect on the binding of FAP-1 to Fas as achieved with the 4-15 synthetic peptides. Furthermore, Fas/FAP-1 binding was extensively investigated using the scanned tripeptides to determine the critical amino acids residues required for inhibition (FIG. 3C). The results revealed that the third amino acids residues from the C-terminus, and the C-terminal amino acids having the strongest inhibitory effect were either serine or threonine; and either valine, leucine, or isoleucine, respectively. However, there were no differences among the second amino acid residues from the C-terminus with respect to their inhibitory effect on Fas/FAP-1 binding. These results were consistent with those of the yeast two-hybrid system (FIGS. 2C and 2D). Therefore, it was concluded that the C-terminal three amino acids (SLV) are critical determinants of Fas binding to the third PDZ domain of FAP-1 protein.

To further substantiate that the PDZ domain interacts with tS/T-X-V/L/I under more native conditions, GST-fused FAP-1 proteins were tested for their ability to interact with Fas expressed in Jurkat T-cells. The results revealed that the tripeptide Ac-SLV, but not Ac-SLY, abolished in a dose-dependent manner the binding activity of FAP-1 to Fas proteins extracted from Jurkat T-cells (FIGS. 4C and 4D). This suggests that the C-terminal amino acids tSLV are the minimum binding site for FAP-1, and that the amino acids serine and valine are critical for this physical association.

To next examine the hypothesis that the physiological association between the C-terminal three amino acids of Fas and the third PDZ domain of FAP-1 is necessary for the in vivo function of FAP-1 as a negative regulator of Fas-mediated signal transduction, a microinjection experiment was employed with synthetic tripeptides in a colon cancer cell line, DLD-1, which expresses both Fas and FAP-1, and is resistant to Fas-induced apoptosis. The experiments involved the direct microinjection of the synthetic tripeptides into the cytoplasmic regions of single cells and the monitoring of the physiological response to Fas-induced apoptosis in vivo. The results showed that microinjection of Ac-SLV into DLD-1 cells dramatically induced apoptosis in the presence of Fas-monoclonal antibodies (CH11, 500 ng/ml) (FIGS. 5A, 5E and FIG. 6), but that microinjection of Ac-SLY and PBS/K did not (FIGS. 5B, 5F and FIG. 6). These results strongly support the hypothesis that the physical association of FAP-1 with the C-terminus of Fas is essential for protecting cells from Fas-induced apoptosis.

In summary, it was found that the C-terminal SLV of Fas is alone necessary and sufficient for binding to the third PDZ domain of FAP-1. Secondly, it is proposed that the new consensus motif of tS/T-X-V/L/I for such binding to the PDZ domain, instead of tS/T-X-V. It is therefore possible that FAP-1 plays important roles for the modulation of signal transduction pathways in addition to its physical interaction with Fas. Thirdly, it is demonstrated that the targeted induction of Fas-mediated apoptosis in colon cancer cells by direct microinjection of the tripeptide Ac-SLV. Further investigations including the identification of a substrate(s) of FAP-1 and structure-function analysis will provide insight to the potential therapeutic applications of Fas/FAP-1 interaction in cancer as well as provide a better understanding of the inhibitory effect of FAP-1 on Fas-mediated signal transduction.

Second Series of Experiments

FAP-1 was originally identified as a membrane-associated protein tyrosine phosphatase which binds to the C-terminus of Fas, and possesses six PDZ domains (also known as DHR domain or GLGF repeat) PDZ domain has recently been shown as a novel module for specific protein-protein interaction, and it appears to be important in the assembly of membrane proteins and also in linking signaling molecules in a multiprotein complex. In recent comprehensive studies, it was found that the third PDZ domain of FAP-1 specifically recognized the sequence motif t(S/T)-X-V and interacts with the C-terminal three amino acids SLV of Fas (FIG. 9). In order to investigate the possibility that FAP-1 also interacts with the C-terminal region of p75NGFR (FIG. 8), an in vitro binding assay, was performed as well as, a yeast two-hybrid analysis by using a series of deletion mutants of p75NGFR. The results revealed that the C-terminal cytoplasmic region of p75NGFR, which is highly conserved among all species, interacts with FAP-1 (FIG. 10). Furthermore, the C-terminal three amino acids SPV of p75NGFR were necessary and sufficient for the interaction with the third PDZ domain of FAP-1 (FIGS. 11A and 11B). Since FAP-1 expression was found highest in fetal brain, these findings imply that interaction of FAP-1 with p75NGFR plays an important role for signal transduction pathway via p75NGFR in neuronal cells as well as in the formation of the initial signal-transducing complex for p75NGFR.

### REFERENCES

1. Banville, D., et al. J. Biol. Chem. 269: 22320-22327 (1994).

2. Boldin, M. P. et al. J. Biol. Chem. 270: 7795-7798 (1995).

3. Camerini, D., et al. J. Immunol. 147: 3165-3169 (1991).

4. Chao, M. V. and B. L. Hempstead TINS 18: 321-326 (1995).

5. Chinnaiyan, A. M., et al. Cell 81: 505-512 (1995).

6. Cho, K.-O., et al. Neuron 9: 929-942 (1992).

7. Conboy, J. G., et al. J. Biol. Chem. 266: 8273-8280 (1991).

8. Doyle, D. A., et al. Cell 85: 1067-1076 (1996).

9. Funayama, N., et al. J. Cell Biol. 115: 1039-1048 (1991).

10. Gould, K. L., et al. EMBO J. 8: 4133-4142 (1989).

11. Gu, M. X., et al. Proc. Natl. Acad. Sci. U.S.A. 88: 5867-5871 (1991).

12. Hill, D. E., et al. Meth. Enzymol. 155, 558-568 (1987).

13. Ito, N., and Nagata, S. J. Biol. Chem. 268: 10932-10937 (1993).

14. Itoh, N. et al. Cell 66: 233-243 (1991).

15. Johnson, D. et al. Cell 47: 545-554 (1986).

16. Kim, E., et al. Nature 378: 85-88 (1995).

17. Kischkel, F. C. et al. EMBO J. 14: 5579-5588 (1995).

18. Kitamura, K. et al. FEBS Lett. 351: 35-37 (1994).

19. Kornau, H.-C., et al. Science 269:1737-1740 (1995).

20. Lankes, W. T., and Furthmayr, H. Proc. Natl. Acad. Sci. U.S.A. 88: 8297-8301 (1991).

21. Maekawa, K., et al. FEBS Letters 337: 200-206 (1994).

22. Mallett, S., et al. EMBO J. 9: 1063-1068 (1990).

23. Matsumine, A. et al. Science 272: 1020-1023 (1996)

24. McGahon, A. J. et al. Meth. Cell Biol. 46: 153-185 (1995).

25. Pantel, K. et al. J. Natl. Cancer Inst. 87: 1162-1168 (1995).

26. Rouleau, G. et al. Nature 363: 515-521 (1993).

27.

28. Sambrook, J., et al. (1989) Molecular Cloning: a laboratory manual. Second Edition. Cold Spring Harbor Laboratory Press.

29. Sato, T., et al. Science 268: 411-415 (1995).

30. Schnorrenberg, G. and Gerhardt H. Tetrahedron 45: 7759-7764 (1989).

31. Saras, J., et al. J. Biol. Chem. 269, 24082-24089 (1994).

32. Smith, C. A. et al. Cell 73: 1349-1360 (1993).

33. Stamenkovic, I., et al. EMBO J. 8: 1403-1410 (1989).

34. Stanger, B. Z., et al. Cell 81: 513-523 (1995).

35. Takahashi, T. et al. Cell 76: 969-976 (1994).

36. Vogel, W., et al. (1993). Science 259: 1611-1614 (1993).

37. Watanabe-Fukunaga, R., et al. Nature 356: 314-317 (1992).

38. Wang, X. W., et al. Cancer Res. 55: 6012-6016 (1995).

39. Westendorp, M. O. et al. Nature 375: 497-500 (1995).

40. Woods, D. F. and Bryant, P. J. Cell 66: 451-464 (1991).

41. Yang, Q., and Tonks, N. K. Proc. Natl. Acad. Sci. U.S.A. 88: 5949-5953 (1991).

What is claimed is:

1. A composition capable of inhibiting specific binding between a signal-transducing protein and a cytoplasmic protein containing the amino acid sequence (G/S/A/E)-L-G-(F/I/L), wherein each—represents a peptide bond, each parenthesis encloses amino acids which are alternatives to one other, and each slash within such parentheses separating the alternative amino acids.

2. The composition of claim 1, wherein the cytoplasmic protein contains the amino acid sequence (K/R/Q)-Xₙ-(G/S/A/E)-L-G-(F/I/L), wherein X represents any amino acid which is selected from the group comprising the twenty naturally occurring amino acids and n represents at least 2, but not more than 4.

3. The composition of claim 1, wherein the cytoplasmic protein contains the amino acid sequence SLGI.

4. The composition of claim 1, wherein the signal-transducing protein has at its carboxyl terminus the amino acid sequence (S/T)-X-(V/I/L), wherein each—represents a peptide bond, each parenthesis encloses amino acids which are alternatives to one other, each slash within such parentheses separating the alternative amino acids, and the X represents any amino acid which is selected from the group comprising the twenty naturally occurring amino acids.

5. The composition of claim 1, wherein the composition comprises an antibody, an inorganic compound, an organic compound, a peptide, a peptidomimetic compound, a polypeptide, or a protein.

6. The composition of claim 5, wherein the peptide comprises the sequence (S/T)-X-(V/I/L)-COOH, wherein each—represents a peptide bond, each parenthesis encloses amino acids which are alternatives to one other, each slash within such parentheses separating the alternative amino acids, the X represents any amino acid which is selected from the group comprising the twenty naturally occurring amino acids.

7. The composition of claim 6, wherein the peptide has the amino acid sequence DSENSNFRNEIQSLV.

8. The composition of claim 6, wherein the peptide has the amino acid sequence RNEIQSLV.

9. The composition of claim 6, wherein the peptide has the amino acid sequence NEIQSLV.

10. The composition of claim 6, wherein the peptide has the amino acid sequence EIQSLV.

11. The composition of claim 6, wherein the peptide has the amino acid sequence IOSLV.

12. The composition of claim 6, wherein the peptide has the amino acid sequence QSLV.

13. The composition of claim 6, wherein the peptide has the amino acid sequence SLV.

14. The composition of claim 6, wherein the peptide has the amino acid sequence IPPDSEDGNEEQSLV.

15. The composition of claim 6, wherein the peptide has the amino acid sequence DSEMYNFRSQLASVV.

16. The composition of claim 6, wherein the peptide has the amino acid sequence IDLASEFLFLSNSFL.

17. The composition of claim 6, wherein the peptide has the amino acid sequence PPTCSQANSGRISTL.

18. The composition of claim 6, wherein the peptide has the amino acid sequence SDSNMNMNELSEV.

19. The composition of claim 6, wherein the peptide has the amino acid sequence QNFRTYIVSFV.

20. The composition of claim 6, wherein the peptide has the amino acid sequence RETIESTV.

21. The composition of claim 6, wherein the peptide has the amino acid sequence RGFISSLV.

22. The composition of claim 6, wherein the peptide has the amino acid sequence TIQSVI.

23. The composition of claim 6, wherein the peptide has the amino acid sequence ESLV.

24. The composition of claim 6, wherein the organic compound has the sequence Ac-SLV-COOH, wherein the Ac represents an acetyl, each—represent a peptide bond.

25. A composition capable of inhibiting specific binding between a signal-transducing protein having at its carboxyl terminus the amino acid sequence (S/T)-X-(V/I/L), wherein each—represents a peptide bond, each parenthesis encloses amino acids which are alternatives to one other, each slash within such parentheses separating the alternative amino acids, the X represents any amino acid which is selected from the group comprising the twenty naturally occurring amino acids, and a cytoplasmic protein.

26. The composition of claim 25, wherein the composition comprises an antibody, an inorganic compound, an organic compound, a peptide, a peptidomimetic compound, a polypeptide or a protein.

27. A method of identifying a compound capable of inhibiting specific binding between a signal-transducing protein and a cytoplasmic protein containing the amino acid sequence (G/S/A/E)-L-G-(F/I/L), wherein each—represents a peptide bond, each parenthesis encloses amino acids which are alternatives to one other, each slash within such parentheses separating the alternative amino acids, which comprises:
(a) contacting the cytoplasmic protein bound to the signal-transducing protein with a plurality of compounds under conditions permitting binding between a known compound previously shown to be able to displace the signal-transducing protein bound to the cytoplasmic protein and the bound cytoplasmic protein to form a complex; and
(b) detecting the displaced signal-transducing protein or the complex formed in step (a), wherein the displacement indicates that the compound is capable of inhibiting specific binding between the signal-transducing protein and the cytoplasmic protein.

28. The method of claim 27, wherein the inhibition of specific binding between the signal-transducing protein and the cytoplasmic protein affects the transcription activity of a reporter gene.

29. The method of claim 28, where in step (b) the displaced signal-transducing protein or the complex is detected by comparing the transcription activity of a reporter gene before and after the contacting with the compound in step (a), where a change of the activity indicates that the specific binding between the signal-transducing protein and the cytoplasmic protein is inhibited and the signal-transducing protein is displaced.

30. The method of claim 27, wherein the cytoplasmic protein is bound to a solid support.

31. The method of claim 27, wherein the compound is bound to a solid support.

32. The method of claim 27, wherein the compound comprises an antibody, an inorganic compound, an organic compound, a peptide, a peptidomimetic compound, a polypeptide or a protein.

33. The method of claim 27, wherein the contacting of step (a) is in vitro.

34. The method of claim 27, wherein the contacting of step (a) is in vivo.

35. The method of claim 34, wherein the contacting of step (a) is in a yeast cell.

36. The method of claim 34, wherein the contacting or step (a) is in a mammalian cell.

37. The method of claim 27, wherein the signal-transducing protein is a cell surface receptor.

38. The method of claim 27, wherein the signal-transducing protein is a signal transducer protein.

39. The method of claim 27, wherein the signal-transducing protein is a tumor suppressor protein.

40. The method of claim 37, wherein the cell surface protein is the Fas receptor.

41. The method of claim 40, wherein the Fas receptor is expressed in cells derived from organs comprising the thymus, liver, kidney, colon, ovary, breast, testis, spleen, stomach, prostate, uterus, skin, head and neck.

42. The method of claim 40, wherein the Fas receptor is expressed in cells comprising T-cells and B-cells.

43. The method of claim 37, wherein the cell-surface receptor is the CD4 receptor.

44. The method of claim 37, wherein the cell-surface receptor is the p75 receptor.

45. The method of claim 37, wherein the cell-surface receptor is the serotonin 2A receptor.

46. The method of claim 37, wherein the cell-surface receptor is the serotonin 2B receptor.

47. The method of claim 38, wherein the signal transducer protein is Protein Kinase-C-α-type.

48. The method of claim 39, wherein the tumor suppressor protein is adenomatosis polyposis coli tumor suppressor protein.

49. The method of claim 39, wherein the tumor suppressor protein protein is the colorectal mutant cancer protein.

50. The method of claim 27, wherein the cytoplasmic protein contains the amino acid sequence SLGI, wherein each—represents a peptide bond, each parenthesis encloses amino acids which are alternatives to one other, and each slash within such parentheses separating the alternative amino acids.

51. The method of claim 40, wherein the cytoplasmic protein is Fas-associated phosphatase-1.

52. A method of identifying a compound capable of inhibiting specific binding between a signal-transducing protein having at its carboxyl terminus the amino acid sequence (S/T)-X-(V/I/L), wherein each—represents a peptide bond, each parenthesis encloses amino acids which are alternatives to one other, each slash within such parentheses separating the alternative amino acids, the X represents any amino acid which is selected from the group comprising the twenty naturally occurring amino acids, and a cytoplasmic protein, which comprises:
(a) contacting the signal-transducing protein bound to the cytoplasmic protein with a plurality of compounds under conditions permitting binding between a known compound previously shown to be able to displace the cytoplasmic protein bound to the signal-transducing protein and the bound signal-transducing protein to form a complex; and
(b) detecting the displaced cytoplasmic protein or the complex of step (a) wherein the displacement indicates that the compound is capable of inhibiting specific binding between the signal-transducing protein and the cytoplasmic protein.

53. The method of claim 52, wherein the inhibition of specific binding between the signal-transducing protein and the cytoplasmic protein affects the transcription activity of a reporter gene.

54. The method of claim 53, where in step (b) the displaced cytoplasmic protein or the complex is detected by comparing the transcription activity of a reporter gene before and after the contacting with the compound in step (a), where a change of the activity indicates that the specific binding between the signal-transducing protein and the cytoplasmic protein is inhibited and the cytoplasmic protein is displaced.

55. The method of claim 52, wherein the cytoplasmic protein is bound to a solid support.

56. The method of claim 52, wherein the compound is bound to a solid support.

57. The method of claim 52, wherein the compound comprises an antibody, an inorganic compound, an organic compound, a peptide, a peptidomimetic compound, a polypeptide or a protein.

58. The method of claim 52, wherein the contacting of step (a) is in vitro.

59. The method of claim 52, wherein the contacting of step (a) is in vivo.

60. The method of claim 59, wherein the contacting of step (a) is in a yeast cell.

61. The method of claim 59, wherein the contacting or step (a) is in a mammalian cell.

62. The method of claim 52, wherein the signal-transducing protein is a cell surface receptor.

63. The method of claim 52, wherein the signal-transducing protein is a signal transducer protein.

64. The method of claim 52, wherein the signal-transducing protein is a tumor suppressor protein.

65. The method of claim 62, wherein the cell surface protein is the Fas receptor.

66. The method of claim 65, wherein the Fas receptor is expressed in cells derived from organs comprising the thymus, liver, kidney, colon, ovary, breast, testis, spleen, stomach, prostate, uterus, skin, head and neck.

67. The method of claim 65, wherein the Fas receptor is expressed in cells comprising T-cells and B-cells.

68. The method of claim 62, wherein the cell-surface receptor is the CD4 receptor.

69. The method of claim 62, wherein the cell-surface receptor is the p75 receptor.

70. The method of claim 62, wherein the cell-surface receptor is the serotonin 2A receptor.

71. The method of claim 62, wherein the cell-surface receptor is the serotonin 2B receptor.

72. The method of claim 63, wherein the signal transducer protein is Protein Kinase-C-α-type.

73. The method of claim 64, wherein the tumor suppressor protein is adenomatosis polyposis coli tumor suppressor protein.

74. The method of claim 64, wherein the tumor suppressor protein is the colorectal mutant cancer protein.

75. The method of claim 52, wherein the cytoplasmic protein contains the amino acid sequence SLGI, wherein each—represents a peptide bond, each parenthesis encloses amino acids which are alternatives to one other, and each slash within such parentheses separating the alternative amino acids.

76. The method of claim 52, wherein the cytoplasmic protein is Fas-associated phosphatase-1.

77. A method inhibiting the proliferation of cancer cells comprising the composition of claim 1.

78. The method of claim 77, wherein the cancer cells are derived from organs comprising the thymus, liver, kidney, colon, ovary, breast, testis, spleen, stomach, prostate, uterus, skin, head and neck.

79. The method of claim 77, wherein the cancer cells are derived from cells comprising T-cells and B-cells.

80. A method of inhibiting the proliferation of cancer cells comprising the composition of claim 25.

81. The method of claim 80, wherein the cancer cells are derived from organs comprising the thymus, liver, kidney, colon, ovary, breast, testis, spleen, stomach, prostate, uterus, skin, head and neck.

82. The method of claim 80, wherein the cancer cells are derived from cells comprising T-cells and B-cells.

83. A method of inhibiting the proliferation of cancer cells comprising the compound identified by the method of claim 27.

84. The method of claim 83, wherein the cancer cells are derived from organs comprising the thymus, liver, kidney, colon, ovary, breast, testis, spleen, stomach, prostate, uterus, skin, head and neck.

85. The method of claim 83, wherein the cancer cells are derived from cells comprising T-cells and B-cells.

86. A method of inhibiting the proliferation of cancer cells comprising the compound identified by the method of claim 52.

87. The method of claim 86, wherein the cancer cells are derived from organs comprising the thymus, liver, kidney, colon, ovary, breast, testis, spleen, stomach, prostate, uterus, skin, head and neck.

88. The method of claim 86, wherein the cancer cells are derived from cells comprising T-cells and B-cells.

89. A method of treating cancer in a subject which comprises introducing to the subject's cancerous cells an amount of the composition of claim 1 effective to result in apoptosis of the cells.

90. The method of claim 89, wherein the cancer cells are derived from organs comprising the thymus, liver, kidney, colon, ovary, breast, testis, spleen, stomach, prostate, uterus, skin, head and neck.

91. The method of claim 89, wherein the cancer cells are derived from cells comprising T-cells and B-cells.

92. A method of treating cancer in a subject which comprises introducing to the subject's cancerous cells an amount of the composition of claim 25 effective to result in apoptosis of the cells.

93. The method of claim 92, wherein the cancer cells are derived from organs comprising the thymus, liver, kidney, colon, ovary, breast, testis, spleen, stomach, prostate, uterus, skin, head and neck.

94. The method of claim 92, wherein the cancer cells are derived from cells comprising T-cells and B-cells.

95. A method of treating cancer in a subject which comprises introducing to the subject's cancerous cells an amount of the compound identified by the method of claim 27 effective to allow apoptosis of the cells.

96. The method of claim 95, wherein the cancer cells are derived from organs comprising the thymus, liver, kidney, colon, ovary, breast, testis, spleen, stomach, prostate, uterus, skin, head and neck.

97. The method of claim 95, wherein the cancer cells are derived from cells comprising T-cells and B-cells.

98. A method of treating cancer in a subject which comprises introducing to the subject's cancerous cells an amount of the compound identified by the method of claim 52 effective to result in apoptosis of the cells.

99. The method of claim 98, wherein the cancer cells are derived from organs comprising the thymus, liver, kidney, colon, ovary, breast, testis, spleen, stomach, prostate, uterus, skin, head and neck.

100. The method of claim 98, wherein the cancer cells are derived from cells comprising T-cells and B-cells.

101. A method of inhibiting the proliferation of virally infected cells comprising the composition of claim 1.

102. A method of inhibiting the proliferation of virally infected cells comprising the composition of claim 25.

103. A method of inhibiting the proliferation of virally infected cells comprising the compound identified by the method of claim 27.

104. A method of inhibiting the proliferation of virally infected cells comprising the compound identified by the method of claim 52.

105. The method of claim 101, wherein the virally infected cells comprise Hepatitis B virus, Epstein-Barr virus, influenza virus, Papilloma virus. Adeno virus, Human T-cell lymphtropic virus, type 1 or HIV.

106. The method of claim 102, wherein the virally infected cells comprise Hepatitis B virus, Epstein-Barr virus, influenza virus, Papilloma virus. Adeno virus, Human T-cell lymphtropic virus, type 1 or HIV.

107. The method of claim 103, wherein the virally infected cells comprise Hepatitis B virus, Epstein-Barr virus, influenza virus, Papilloma virus. Adeno virus, Human T-cell lymphtropic virus, type 1 or HIV.

108. The method of claim 104, wherein the virally infected cells comprise Hepatitis B virus, Epstein-Barr virus, influenza virus, Papilloma virus. Adeno virus, Human T-cell lymphtropic virus, type 1 or HIV.

109. A method of treating a virally-infected subject which comprises introducing to the subject's virally-infected cells the composition of claim 1 effective to result in apoptosis of the cells.

110. A method of treating a virally-infected subject which comprises introducing to the subject's virally infected cells the composition of claim 25 effective to result in apoptosis of the cells.

111. A method of treating a virally-infected subject which comprises introducing to the subject's virally-infected cells an amount of the compound identified by the method of claim 27 effective to result in apoptosis of the cells.

112. A method of treating a virally-infected subject which comprises introducing to the subject's virally-infected cells an amount of the compound identified by the method of claim 52 effective to result in apoptosis of the cells.

113. The method of claim 109, wherein the virally infected cells comprise the Hepatitis B virus, Epstein-Barr virus, influenza virus, Papilloma virus. Adeno virus, Human T-cell lymphtropic virus, type 1 or HIV.

114. The method of claim 110, wherein the virally infected cells comprise the Hepatitis B virus, Epstein-Barr virus, influenza virus, Papilloma virus. Adeno virus, Human T-cell lymphtropic virus, type 1 or HIV.

115. The method of claim 111, wherein the virally infected cells comprise the Hepatitis B virus, Epstein-Barr virus, influenza virus, Papilloma virus. Adeno virus, Human T-cell lymphtropic virus, type 1 or HIV.

116. The method of claim 112, wherein the virally infected cells comprise the Hepatitis B virus, Epstein-Barr virus, influenza virus, Papilloma virus. Adeno virus, Human T-cell lymphtropic virus, type 1 or HIV.

117. A pharmaceutical composition comprising the composition of claim 1 in an effective amount and a pharmaceutically acceptable carrier.

118. A pharmaceutical composition comprising the composition of claim 25 in an effective amount and a pharmaceutically acceptable carrier.

119. A pharmaceutical composition comprising the compound identified by the method of claim 27 in an effective amount and a pharmaceutically acceptable carrier.

120. A pharmaceutical composition comprising the compound identified by the method of claim 52 in an effective amount and a pharmaceutically acceptable carrier.
